(19) United States
(12) Patent Application Publication
(10) Pub. No.: US 2009/0275788 A1
(43) Pub. Date: Nov. 5, 2009

(54) Hydrogenation Processes Using Functional Surface Catalyst Composition

(75) Inventors: Robert L Bedard, Des Plaines, IL (US); Jeffery C. Bricker, Des Plaines, IL (US); Dean E. Rende, Des Plaines, IL (US); Ally S. Chan, Des Plaines, IL (US)

Correspondence Address: HONEYWELL/UOP;PATENT SERVICES, 101 COLUMBIA DRIVE, P O BOX 2245 MAIL STOP AB/2B, MORRISTOWN, NJ, 07962, US

(73) Assignee: UOP LLC, DES PLAINES (IL)

(21) Appl. No.: 12/447,096

(22) PCT Filed: Nov. 9, 2007
(86) PCT No.: PCT/US07/84198

§ 371 Date: Apr. 24, 2009

Related U.S. Application Data
(60) Provisional application No. 60/865,411, filed on Nov. 11, 2006.

(51) Int. Cl. C07C 5/10

(52) U.S. Cl. 585/270; 585/266; 585/269

(57) ABSTRACT

Hydrogenation processes using a catalyst composition which, preferably comprises a glass substrate, with one or more functional surface active constituents integrated on and/or in the substrate surface. A substantially nonporous substrate has (i) a total surface area between about 0.01 m²/g and 10 m²/g; and (ii) a predetermined isoelectric point (IEP) obtained in a pH range greater than 0, preferably greater than or equal to 4.5, or more preferably greater than or equal to 6.0, but less than or equal to 14. At least one catalytically-active region may be contiguous or discontiguous and has a mean thickness less than or equal to about 30 nm, preferably less than or equal to 20 nm and more preferably less than or equal to 10 nm. Preferably, the substrate is a glass composition having a SARC_{Na} less than or equal to about 0.5.

### FIELD OF THE INVENTION

This invention relates to a catalyst composition, and its method of making and manufacture, useful for a diversity of chemical production processes as well as various emission control processes. More specifically, it relates to a catalyst composition, preferably comprising a glass substrate, with one or more functional surface active constituents integrated on and/or in the substrate surface, which can be used in a diversity of hydrogenation process applications.

### BACKGROUND OF THE INVENTION

Catalyst compositions are used to promote a class of chemical reactions generally described as catalytic reactions or catalysis. Catalysis is important to efficiently operating a wide range of chemical processes.

Most industrial reactions and nearly all biological reactions are either catalytic or involve pre- or post-reaction treatments that are catalytic. The value of the products made in the United States alone in processes that, at some stage, involve catalysis approaches about one trillion dollars (USD). Products made with catalyst compositions include, for example, food, clothing, pharmaceuticals, commodity chemicals, specialty or fine chemicals, plastics, detergents, fuels and lubricants, among others. Catalyst compositions are also useful for treating emissions (e.g., auto emissions, refinery emissions, utility plant emissions, etc.) and other process discharge streams for reducing the content of potentially harmful components that could adversely affect individual health or the environment.

In terms of market sales, solid, supported catalysts, used in heterogeneous catalysis reactions, represent about $3 billion/year worldwide market. Solid, supported catalysts generally fall in three groups, petroleum refining, chemical processing and emission control catalysts. Between these three classes of catalyst markets, sales are roughly split in thirds. For example, in 1990, of the $1.8 billion U.S. solid catalyst market, petroleum refining, chemical processing and emission control catalysts comprised 37%, 34% and 29% of the market, respectively. And of the petroleum refining catalyst market, for example, (about $1 billion in 1990) 56% of revenue came from fluid catalytic cracking (FCC) catalysts, while 31.5%, 6.5% and 4.5% of revenue came from hydrotreating, hydrocracking and reforming catalysts, respectively.

From a chemical mechanism standpoint, without being substantially consumed itself, a catalyst generally works to increase the rate at which a chemical reaction reaches a state of equilibrium between reactants and products. So, although a catalyst cannot alter the state of equilibrium between reactants and product, for any given reaction of interest, it can, if properly designed and/or selected, accelerate the rate of chemical reactions.

Consequently, catalysts are used in a wide range of commercially useful processes for an array of purposes including improving the reactivity, selectivity, and energy efficiency of the process, among other purposes. For example, improving the rate of reaction or reactivity of reactants to produce the desired product(s) under specified process conditions can reduce processing time, so higher product throughputs (e.g., increased product volume or mass per unit hour) can be obtained. So, catalyst activity indicates the catalyst composition's ability to effectively convert reactants to the desired product(s) per unit time. Similarly, improving reaction selectivity can improve the percentage yield of desired product(s) across a range of possible, reaction products, some of which may be undesired and require further processing to either remove or convert, accordingly. So, catalyst selectivity is the catalyst composition's ability to convert a fraction of reactant(s) to a particular product under specified process conditions. In addition, catalyst compositions can be used to convert and reduce levels of contaminants or undesired reactants or products in a process. And still another purpose is to improve the overall energy efficiency of the reaction process, while either maintaining or improving product throughputs and/or reaction selectivity.

The scale at which catalysts can be used can vary widely. For example, without limitation, catalysts can be used for reducing pollutant levels such as hydrocarbons, carbon monoxide (CO), nitrogen oxides (NOₓ) and sulfur oxides (SOₓ), which may be found in the emissions for a range of processes, from gasoline or diesel combustion exhausts of vehicles to assorted petroleum refining or coal-burning processes. Similarly, catalysts can be used in hydrocarbon treatment processes used for converting or modifying hydrocarbon process streams from many different sources including, for example, virgin petroleum fractions, recycle petroleum fractions, heavy oil, bitumen, shale, natural gas, among other carbon containing materials susceptible to catalytic reactions.

Catalytic reactions generally fall in one of two distinct classes of reaction types—homogeneous catalysis and heterogeneous catalysis.

Homogeneous catalysis broadly describes a class of catalytic reactions in which the reactants and catalyst are mixed together in a solution-phase, which is typically a liquid-phase system, though gas-phase catalytic reactions have been used in some cases. Consequently, concentration gradients and the transport of the reactants to the catalyst can become important considerations in controlling a homogeneous catalytic reaction. Also, in some instances “solution-phase” catalytic reactions can occur across the interface of two liquid phases, not forming a true solution, but rather an emulsion phase. Some general categories of homogeneous catalysis include acid-base catalysis, organometallic catalysis and phase-transfer catalysis, among others.

Heterogeneous catalysis, on the other hand, describes a class of catalytic reactions in which the reactants, in either a gas or liquid phase, are exposed to a catalyst that's in a substantially solid or semi-solid phase. So, in heterogeneous catalysis, the catalyst and reactants produce a mixed solid-liquid or solid-gas phase reaction. Heterogeneous catalysis has a number of advantages versus homogeneous catalysis including, for example, the tendency for solid catalysts to (a) be less corrosive and hence present relatively lower safety and environmental risks versus many homogeneous solution-phase catalysts, (b) allow a wider range of economically viable temperature and pressure conditions and (c) allow better control of more strongly exothermic and endothermic chemical reactions, among other advantages.

On the other hand, a solid can have mass transport limitations that could significantly reduce the catalyst's ultimate effectiveness. Typically, a solid catalyst (or catalyst particle, as it's sometimes called) comprises one or more catalytic constituents (e.g., a noble metal such as Pd, Pt, Ru, Re, etc.) on a porous material with very high internal surface areas, usually on the order of hundreds of square meters per gram, where the catalytic constituent resides. So a conventional catalyst composition or catalyst particle includes a particularly porous support with high internal surface area where the catalytic reaction occurs. However, this type of catalyst structure can, and often does, create a mass transport limitation that can reduce the catalyst particle's effective performance, both with respect to catalyst activity and selectivity, among other catalyst performance issues.

In this more typical catalyst structure, reactants must diffuse through the network of pores, to reach the catalyst particle's internal area and the product(s) must diffuse back out. Accordingly then, the extent of a conventional catalyst composition's porosity is determined by balancing, among other things, the trade-off between two properties of conventional catalyst compositions, namely, catalyst surface area versus ability to facilitate mass transport. For instance, many catalytic constituents typically reside on a support with a fine and intricate pore structure, often micropores (i.e., <2 nm mean maximum diameter), to increase the catalyst particle's surface area. This higher surface area, in turn, will normally produce an increase in catalyst activity. But the gain in catalyst activity, arising from higher catalyst particle surface area, usually induces a problem with resistance to mass transport (i.e., movement of reactants and product in and out of the catalyst particle), particularly where the support comprises a significant percentage of micropore structure. Reducing resistance to mass transport (i.e., faster mass transport) could be addressed by increasing the percentage of larger size pores (e.g., macropores, >50 nm) in the support. However, that solution, in turn, tends to reduce the catalyst particle's physical strength and durability. Put another way, the catalyst particle becomes less robust, from a mechanical standpoint.

Meanwhile, if reactant(s) confront significant mass transport resistance in the catalyst particle's pore structure, a concentration gradient will exist under steady state reaction conditions. Consequently, the concentration of the reactant(s) in the pore structure is a maximum at the catalyst particle's periphery and minimum at its center. On the other hand, the reaction product concentration will be higher at the catalyst particle's center than at its periphery. These concentration gradients provide the driving force for the transport. The larger these concentration gradients become, the lower the rate of the catalytic reaction becomes. In turn, the catalyst particle's effective performance (e.g., reactivity, selectivity, life cycle between regeneration treatments, resistance to coking, etc.) is reduced, accordingly.

Generally, catalyst compositions are developed to improve on one or more processing objectives like those noted above from a commercial standpoint. In some cases, one factor affecting catalyst performance is its ability to promote a rapid, but effective, reaction between reactants. Accordingly, a catalyst composition with reduced diffusion limitations is frequently desired. In other instances, however, selectivity towards producing particular products may be relatively more important so that the preferred product(s) are obtained. In turn, additional process steps and related processing equipment, used to remove or convert undesired reaction products, may be eliminated.

For example, in 1976 Y. T. Shah et al. proposed the use of acid-leached aluminoborosilicate fibers, specifically E-glass (more specifically, E-621) to produce a catalyst support with a higher surface area to volume ratio than conventional catalysts to reduce the size of a catalytic converter for an auto emission system (see e.g., Oxidation of an Automobile Exhaust Gas Mixture by Fiber Catalysts, Ind. Eng. Chem., Prod. Res. Dev., pp. 29-35, Vol. 15, No. 1, 1976.) At the same time, Shah et al. believed the higher surface area produced in the leached E-glass would be readily accessible to reactant gases typically produced in an auto exhaust gas mixture (e.g., CO, CO₂, NOₓ, methane, ethane, propane, ethylene, propylene, acetylene, benzene, toluene, etc.).

As compared to two conventional catalysts, Pt supported by either alumina beads or silica gel beads, Shah et al. showed that a smaller amount of fiber E-glass catalyst carrier with comparatively lower surface area (75 m²/g) performed better versus the alumina supported or silica supported catalysts (180 m²/g and 317 m²/g, respectively), where the average pore size of the E-glass catalyst was larger versus either the alumina or silica supported catalysts. Nonetheless, Shah et al. did not propose or suggest that effective auto exhaust oxidation could occur at surface areas below 75 m²/g.

Nearly 25 years later, in 1999, Kiwi-Minsker et al. studied the effect of reduced surface area in another leached aluminoborosilicate E-glass fiber (EGF) versus a silica glass fiber (SGF) used in selective liquid-phase hydrogenation of benzaldehyde to produce either benzyl alcohol (with a Pt-based catalyst) or toluene (with a Pd-based catalyst) (see e.g., Supported Glass Fibers Catalysts for Novel Multi-phase Reactor Design, Chem. Eng. Sci. pp. 4785-4790, Vol. 54, 1999). In that study, Kiwi-Minsker et al. found that the SGF was not susceptible to obtaining an increased surface area from acid-leaching so its surface area remained low at 2 m²/g versus EGF sample surface areas of 15 m²/g and 75 m²/g, respectively, used for supporting Pd as a catalytic constituent for a Pd-based catalyst composition. But Kiwi-Minsker et al. noted that the SGF/Pd catalyst had substantially the same effective surface concentration of Pd (millimoles of metal per m²) as its EGF/Pd catalyst counterparts (i.e., about 0.1 mmol/m²) and yet the SGF/Pd catalyst composition demonstrated a lower activity or reaction rate per gram of Pd vs. its EGF/Pd catalyst counterparts.

Kiwi-Minsker et al. suggested that this lower activity for the lower surface area SGF/Pd catalyst may be explained by a stronger interaction of the active component (i.e., catalytic constituent, Pd in this case) with the SGF support, rather than its lower surface area (i.e., 2 m²/g). However, they failed to validate this point by demonstrating that an EGF/Pd catalyst, with a yet lower surface area (i.e., comparable to the SGF/Pd at 2 m²/g) was, at least, as catalytically as active as the EGF/Pd catalyst samples with higher surface areas (i.e., 15 m²/g and 75 m²/g, respectively). Accordingly, it's unclear that the reason for SGF/Pd's activity limitation, which Kiwi-Minsker et al. suggest—namely, a stronger interaction between Pd and the SGF, due to SGF's higher acidity vs. EGF—is the dominant factor, rather than the SGF/Pd's substantially lower surface area. In any case, Kiwi-Minsker did not report an improved rate of diffusion, and hence, catalytic activity, for the 15 m²/g EGF/Pd sample versus the 75 m²/g EGF/Pd sample, which might have otherwise suggested a beneficial effect arising from a lower catalyst surface area.

More recently, in U.S. Pat. No. 7,060,651 and EP 1 247 575 A1 (EP '575) Barelko et al. disclose the beneficial effects of using a silica-rich support, comprising silicon oxide and nonsilica-containing oxides (e.g., Al₂O₃, B₂O₃, Na₂O, MgO, CaO, etc.), as a catalyst support, wherein the silica-rich support has pseudo-layered microporous structures in the sub surface layers of the support (see e.g., par. 11, 13, 15, 17, 18, 23, 31 and 32 of EP '575). As explained more fully to the European Patent Office (“EPO”), in distinguishing EP '575 over the catalytic supports disclosed in the Kiwi-Minsker et al. paper noted above (“Kiwi-Minsker supports”), Barelko et al. asserted that their claimed silica-rich supports have pseudo-layered microporous structures with narrow interlayer spaces, while the Kiwi-Minsker supports do not. More specifically, Barelko et al. argued that there are no grounds in the Kiwi-Minsker et al. paper to suppose that (a) pseudo-layered microporous structures with narrow interlayer spaces are formed in the Kiwi-Minsker supports and (b) such pseudo-layered microporous structures with narrow interlayer spaces are responsible for enhancing the activity of the metal applied to the support (see e.g., par. 13, 17-18, 23 and 32 of EP '575).

Barelko et al. further distinguished its silica-rich supports over Kiwi-Minsker et al. by explaining to the EPO that their support's more highly active catalytic state arises from “a predominant distribution of the catalytic components in the sub-surface layers of the support in a highly dispersed active state” (underscoring in original text), which, in turn, make the catalytic components resistant to sintering, agglomeration, peeling off of the support and the effects of contact poisons (see e.g., par. 11 of EP '575). EP '575 acknowledges that diffusion restrictions may retard incorporating cations into the support's interlayer spaces, and hence, cation chemisorption into the support (see e.g., par. 17 of EP '575). To overcome this diffusion restriction problem, Barelko et al. proposed (and claimed) a support structure in which “thin” layers of Si—O fragments are separated to form narrow interlayer spaces (i.e., pseudo-layered microporous structure) containing a “large number” of OH groups whose protons can be cation exchanged. Barelko et al. disclose that sufficiently “thin” layers of Si—O fragments are characteristic of a high Q³ to Q⁴ ratio and further assert that the pseudo-layered microporous structures, with a large number of OH groups sandwiched between the narrow interlayer spaces, are confirmed by ²⁹Si NMR and IR spectroscopic measurements in combination with argon BET and alkali titration surface area measurements.

Like some of these glass catalyst compositions, many conventional catalysts endeavor to address at least one of the above-identified processing issues, but which can fall short in some other aspect of catalyst performance. So, they are frequently restricted to a relatively narrow range of process reactions, have limited cycle of use before requiring regeneration or replacement and/or may require significant loadings of costly catalytic constituents (e.g., precious metals such as Pt, Pd, etc.), which can significantly increase the cost of catalyst production as well as operating the catalytic process.

Accordingly, there is a need for an improved catalyst composition that can be used in a variety of processing reactions, while improving process reactivity, selectivity and/or energy efficiency, among other improvements. Preferably, this catalyst composition can provide improvements across a relatively diverse set of process conditions and requirements, while maintaining a relatively higher life cycle with improved robustness and durability. Applicants have discovered a functional surface catalyst composition that is expected to meet this need for wide array of catalytic reactions.

### SUMMARY OF THE INVENTION

According to one aspect of the invention, there is provided a process comprising hydrogenation of a process stream using a catalyst composition to hydrogenate at least a portion of the process stream, the process stream comprising at least one compound having at least one hydrogenatable site, wherein the catalyst composition comprises:
a substantially nonporous substrate having an external surface, a surface region and a subsurface region,
at least one catalytic constituent, and
at least one catalytically-active region, comprising the at least one catalytic constituent, wherein
   a) the substantially nonporous substrate has
i) a total surface area, as measured by a method selected from the group consisting of S.A._{N2-BET}, S.A._{Kr-BET} and combinations thereof, between about 0.01 m²/g and 10 m²/g; and
ii) a predetermined isoelectric point (IEP) obtained in a pH range greater than 0, but less than or equal to 14;
   b) the at least one catalytically-active region may be contiguous or discontiguous and has
i) a mean thickness less than or equal to about 30 nm; and
ii) a catalytically effective amount of the at least one catalytic constituent; and
   c) the location of the at least one catalytically-active region is substantially
i) on the external surface,
ii) in the surface region,
iii) on the external surface in part and in the surface region in part, or
iv) combinations of (c) (i), (ii) and (iii).

Other aspects of the invention will be apparent to those skilled in the art from the following detailed description taken in conjunction with the appended claims and drawings.

### BRIEF DESCRIPTION OF THE DRAWINGS

FIG. 1 is an XPS Sputter Depth Profile corresponding to each of four samples comprising Pd on/in an AR-glass substrate, wherein the Sputter Depth Profile obtained using a PHI Quantum 200 Scanning ESCA Microprobe™ (Physical Electronics, Inc.) with a micro-focused, monochromatized Al Kα X-ray source at 1486.7 eV.

FIG. 2 an XPS Sputter Depth Profile corresponding to each of three samples comprising Pd on/in an A-glass substrate, wherein the Sputter Depth Profile obtained using a PHI Quantum 200 Scanning ESCA Microprobe™ (Physical Electronics, Inc.) with a micro-focused, monochromatized Al Kα X-ray source at 1486.7 eV.

### DETAILED DESCRIPTION OF THE INVENTION

### Definitions

The following terms used herein will have the meaning as defined below.

“Pore” means a cavity or channel that is deeper than it is wide.

“Interconnected Pore” means a pore that communicates with one or more other pores.

“Closed Pore” means a pore without any access to the external surface of the material in which the closed pore is located.

“Open Pore” means a pore with access, whether directly or via another pore or interconnected pore(s), to the external surface of a material in which the open pore is located (i.e., a pore that's not a closed pore).

“Pore Width” means an internal diameter or distance between opposite walls of a pore, as determined by a specified method.

“Pore Volume” means the total volume contribution of all pores excluding the volume contribution of closed pores, as determined by a specified method.

“Porosity” means the ratio of pore volume in a material to the overall volume occupied by the material.

“Micropore” means a pore of internal width less than 2 nanometers (nm).

“Mesopore” means a pore of internal width in the range from 2 nm to 50 nm.

“Macropore” means a pore of internal width greater than 50 nm.

“External Surface” means the external boundary or skin (with a near-zero thickness) of a material including regular or irregular contours associated with defects, if any, on the external boundary or skin.

“Pore Wall Surface” means the internal boundary or skin (with near-zero thickness), including regular or irregular contours associated with defects, if any, on the internal boundary or skin, substantially defining the shape of any open pore in a material having at least one or more types of pore(s).

“Surface” means, collectively, a material's pore wall surface (if any open pores are present), the material's external surface and its surface region.

“Surface Region” means the region of material, excluding any region or regions defined by the material's open pores (if any open pores are present), which may vary depending on the material, but that is (a) less than or equal to 30 nm (preferably, ≦20 nm and more preferably, ≦10 nm) beneath a material's external surface and, to the extent any open pores are present in the material, that is (b) less than or equal to 30 nm (preferably, ≦20 nm and more preferably, ≦10 nm) beneath the material's pore wall surface. For a material with detectable variations in surface elevations, whether regular or irregular, along the external or internal boundary or skin, the average elevation of the external or internal boundary or skin is used for determining an average depth of the surface region.

“Subsurface Region” means the region of a material, excluding any region or regions defined by the material's open pores (if any open pores are present), which may vary depending on the material, but that is (a) greater than 30 nm (preferably, >20 nm and more preferably, >10 nm) beneath the material's external surface and, to the extent any open pores are present in the material, that is (b) greater than 30 nm beneath the material's pore wall surface (preferably, >20 nm and more preferably, >10 nm).

“Internal Surface Area” or “Open Pore Wall Surface Area” means the surface area contribution of all open pore walls in a material, as determined by a specified method.

“External Surface Area” means the surface area contribution of a material excluding the surface area contribution of all pore walls in the material, as determined by a specified method.

“Total Surface Area” means the sum of a material's internal surface area and its external surface area, as determined by a specified method.

“Sodium-Chemisorption Surface Area” or S.A._{Na} means surface area of a material determined by chemisorption of sodium cations using a chemisorption method(s) as described by G. W. Sears Anal. Chem., 1956, vol. 28, p. 1981 and R. Iler, Chemistry of Silica, John Wiley & Sons 1979, p. 203 and 353.

“Sodium-Chemisorption Surface Area Rate of Change” or “SARC_{Na}” where SARC_{Na}=V_{5 to 15}/Vᵢ, wherein, (i) Vᵢ is an initial volume of dilute NaOH titrant solution used to initially titrate an aqueous slurry mixture, comprising a substantially water-insoluble material in a 3.4M NaCl solution at about 25° C., from an initial pH 4.0 to pH 9.0 at time zero, tₒ, and (ii) V_{5 to 15} is the total volume of the same strength NaOH titrant used to maintain the slurry mixture at pH 9 over a 15 minute period, adjusted, as needed and as rapidly as possible, at each of three 5 minute intervals, t₅, t₁₀ and t₁₅, accordingly.

So, Vₜₒₜₐₗ is the total volume of NaOH titrant used over the titration procedure described more fully below, wherein Vᵢ+V_{5 to 15}=Vₜₒₜₐₗ. Accordingly, V_{5 to 15} can also be expressed as the difference between Vₜₒₜₐₗ and Vᵢ, wherein V_{5 to 15}=Vₜₒₜₐₗ−Vᵢ.

For purposes of this definition, the 3.4M NaCl solution is prepared by adding 30 g NaCl (reagent grade) to 150 mL H₂O and 1.5 g of the sample material is added to the NaCl solution to produce an aqueous slurry mixture. The aqueous slurry mixture must be first adjusted to pH 4.0. Either a small amount of dilute acid (e.g., HCl) or base (e.g., NaOH) is used, accordingly, for this adjustment before titration begins with dilute NaOH titrant (e.g., 0.1N or 0.01N) for first obtaining Vᵢ and, thereafter, V_{5 to 15} for making the SARC_{Na} determination. Also, for purposes of this definition, V_{5 to 15} is the cumulative volume of NaOH titrant used at t₅, t₁₀ and t₁₅, wherein the NaOH titrant used is titrated, as rapidly as possible, at each of three 5 minute intervals, to adjust, as needed, the slurry mixture's pH to 9.0 from tₒ to the final time at 15 minutes, t₁₅.

For purposes of this definition, SARC_{Na} is determined for a sample material prior to treatment by any optional ion exchange (IEX), back ion exchange (BIX) and/or electrostatic adsorption (EA) treatment method that may be used for integrating one or more Type-2 constituent precursors (described below) on and/or in the substrate surface.

“Incipient Wetness” means, for an aqueous slurry- or paste-like mixture comprising a solid or semi-solid material for which an isoelectric point (“IEP”) is being determined, the point at which deionized water has substantially covered the entire surface of the solid or semi-solid material and, to the extent present, filled any water-accessible pore volume that the material may have, thereby allowing the water in the aqueous slurry- or paste-like mixture to provide sufficient liquid contact of and between both a glass electrode and its reference-electrode junctions so that the material's IEP can be determined.

“Isoelectric Point” or IEP means the pH at which the net surface charge is zero for a solid or semi-solid material at incipient wetness. IEP, as used herein, may also be referred to as zero point charge (ZPC) or point of zero charge (PZC).

“Catalytically Effective Amount” means a mass of catalytic constituent(s) sufficient to convert, under suitable processing conditions, at least one reactant to at least one predetermined product in sufficient yield to support either a pilot plant or commercial-grade process.

“Chalconide” means a compound containing at least one Group 16 (formerly Group VIA) element from the group consisting of sulfur (S), selenium (Se) and tellurium (Te) and at least one element or radical that's more electropositive than its corresponding Group 16 element.

“Noble Metal” means a transition metal from the group of rhodium (Rh), palladium (Pd), silver (Ag), iridium (Ir), platinum (Pt) and gold (Au), each in a zero oxidation state (while in an unreacted state) unless otherwise indicated as having a charged state in the form of a metal complex, metal salt, metal cation or metal anion.

“Corrosion Resistant Substrate” means a substrate resistant to a substantial alteration in the substrate's compositional structure in its subsurface region, arising from alteration and/or loss of structural constituent elements, new pore production, pore size expansion and the like, by most acids or dilute bases under standard temperature and pressure conditions. However, a corrosion resistant substrate's compositional structure might be substantially altered by high-strength acids (e.g., concentrated HF), bases (e.g., concentrated NaOH) or other highly corrosive reagents, whether alone or in combination with intense temperature, pressure and/or vibrational frequency conditions and still be considered “corrosion resistant” for purposes of this definition.

“Surface Active” means a state in which a material's surface is sufficiently charged with one or more charged constituents to either (i) promote a catalytic reaction under a steady state reaction condition, without further modification, or (ii) otherwise, is adaptable to further modification by either an electrostatic and/or ion exchange interaction with one or more charged constituents, which can subsequently function as catalytic constituent(s) under a steady state reaction condition.

“Substrate” means any solid or semi-solid material, including without limitation, glass and glass-like materials, with an IEP greater than 0 but less than or equal to 14, whose surface active state can be modified, as appropriate, for the substrate's intended use in a catalyst composition having a catalytically effective amount of catalytic constituent(s).

“Integrate” means to associate, for example, a chemical constituent with a substrate through an electronic and/or physicochemical interaction such as, for example, ionic, electrostatic or covalent interactions, including, without limitation, hydrogen bonding, ionic bonding, electrostatic bonding, Van der Waals/dipole bonding, affinity bonding, covalent bonding and combinations thereof.

### DETAILED DESCRIPTION OVERVIEW

The comments under this overview of the detailed description are intended to be only illustrative of selected aspects and factors related to the invention claimed below, and as such, are provided only as a means for conveniently conveying, in brief terms, certain aspects of the detailed description that may be of potential interest to the reader. Accordingly, these overview comments should not be construed to limit the scope of the invention claimed below.

One aspect of the invention relates to a catalyst composition having a surface active catalytically active region(s) having a mean thickness less than or equal to about 30 nm, preferably, ≦ about 20 nm and more preferably, ≦ about 10 nm (“catalyst composition”). Another aspect of the invention relates to various methods of making the novel catalyst composition. Another aspect of the invention is making composited forms of the catalyst composition, whether with or without forming media. Yet another aspect of the invention relates to using the catalyst composition in various processes, such as, for example, hydrocarbon, hetero-hydrocarbon and/or non-hydrocarbon treatment, conversion, refining and/or emission control and treatment processes, among other types of processes. For example, the novel catalyst composition can improve reaction selectivity, reaction rate, product yield and energy efficiency of hydrocarbon, hetero-hydrocarbon and/or non-hydrocarbon treatment, conversion, refining and/or emission control and treatment processes, among other types of processes.

Several factors that should be considered in producing the catalyst composition include, without limitation,
(i) obtaining a substrate with a predetermined isoelectric point (“IEP”), whether as received or after undergoing subsequent treatment(s), in view of the intended use;
(ii) the extent of the substrate's corrosion resistance, in view of the intended use;
(iii) the extent of the substrate's porosity, if any, and related elemental composition, particularly at the surface, for obtaining the desired surface properties, in view of the intended use,
(iv) depending on the composition's intended use, as appropriate, the extent of the substrate's chemical susceptibility to produce a suitable isoelectric point and making it surface active with one or more first constituents having a first type of ionic and/or electrostatic interaction with the substrate that can, but does not necessarily, produce a catalytically active region, having a mean thickness ≦ about 30 nm, preferably, ≦ about 20 nm and more preferably, ≦ about 10 nm, on and/or in the substrate surface;
(v) the substrate's chemical susceptibility to an optional ion exchange (IEX), back ion exchange (BIX) and/or electrostatic adsorption (EA) treatment method for integrating one or more second constituents on and/or in the substrate surface having a second type of ionic and/or electrostatic interaction with the substrate and, accordingly, producing a catalytically active region, having a mean thickness ≦ about 30 nm, preferably, ≦ about 20 nm and more preferably, 5 about 10 nm, on and/or in the substrate surface; and
(vi) depending on the composition's intended use, the treated substrate's chemical susceptibility to, optionally, calcining and/or either reducing, oxidizing, or further chemically reacting the treated substrate with the first or second catalytic constituent prior to using the catalyst composition.

### Substrate Description

### IEP Selection in General & Preferred Range Description for Many Potential Uses

Substrates used for producing a catalyst composition of the invention are preferably glass compositions having an IEP greater than about 0 but less than or equal to 14, whether surface-active, as-received, or treated to produce a surface-active state. Obtaining a substrate with the appropriate IEP suitable for producing a catalyst composition for the intended purpose will depend on a variety of factors, some of which are outlined more generally above (in Detailed Description Overview). Other factors relevant to selecting the appropriate IEP will become more apparent to those skilled in the art in view of the more detailed discussion provided below.

For example, for many processes of commercial interest, glass (or glass-like) compositions and their surface-active products will preferably have an IEP greater than or equal to about 4.5, but less than 14, while glass compositions with an IEP greater than or equal to about 6.0, but less than 14 are often expected to be more preferred and those compositions with an IEP greater than or equal to about 7.8 but less than 14 are often expected to be most preferred. However, depending on the catalyst composition's intended use and the extent and type of porosity in the composition's substrate, the preferred IEP range can be affected. Also, for example, some catalytic processes may be more responsive to a catalyst composition that's surface-active in a lower pH range. Consequently, in those instances a substrate with an IEP less than 7.8, preferably ≦6, and more preferably, ≦4.5, is likely to be more suitable for such processes. So again, it should be understood that selecting a substrate in a suitable IEP range in view of the catalyst composition's intended use will be one factor, in combination with the substrate's porosity, chemical composition and treatment procedures (if any), among other factors.

Again, depending on the intended catalytic use, numerous glass types can be potential substrate candidates for obtaining the suitable IEP and degree and type of porosity, whether as-received, or using one or more of the treatment methods described below. Generally, some examples of such glass types include, without limitation, E-glasses, boron-free E-glasses, S-glasses, R-glasses, AR-glasses, rare earth-silicate glasses, Ba—Ti-silicate glasses, nitrided glasses such as Si-AI-O—N glasses, A-glasses, C-glasses and CC-glasses. However, glass types generally expected to operate for an array of catalytic uses, and selected types of possible treatments are described for illustrative purposes, below.

### AR-Type Glass Description

For example, without limitation, “AR-type” glass is one broad group of substantially nonporous glass compositions with an IEP greater than 7.8. Generally, AR-type glass will contain basic oxide type glass network modifiers in substantial amounts, often 10 wt. % or more of the total glass composition. These basic oxide network modifiers include, for example, without limitation, oxides of Zr, Hf, Al, lanthanides, actinides, alkaline earth oxides (group 2), alkali oxides (group 1), and the like. Zr, Hf, Al, lanthanide, alkaline earth oxide, and alkaline oxide containing glasses are preferred, while Zr containing glass compositions, such as, without limitation, AR-glasses, are particularly preferred.

### A-Type Glass Description

Also, for example, without limitation, “A-type” glass is another broad group of, substantially nonporous glass compositions having an IEP greater than 7.8 but less than 14, whether surface active, as-received, or treated to produce a surface-active state.

Generally, A-type glass will contain either acidic or basic oxide type glass network modifiers including, for example, without limitation, oxides of Zn, Mg, Ca, Al, B, Ti, Fe, Na and K and the like. In the case of basic network modifiers, the amount incorporated in these lower IEP glasses tends to be <12 wt. %. Mg, Ca, Al, Zn, Na and K containing glasses are preferred.

### Non-Leached E-Type Glass Description

Non-leached “E-type” glass is still another non-limiting example of a broad group of substantially nonporous glass compositions having an IEP greater than 7.8 but less than 14, whether surface active, as-received, or treated to produce a surface-active state.

Generally, non-leached E-type glass will contain either acidic or basic oxide type glass network modifiers including, for example, without limitation, oxides of Zn, Mg, Ca, Al, B, Ti, Fe, Na and K and the like. In the case of basic network modifiers, the amount incorporated in these non-leached E-type glasses tends to be <20 wt. %. Mg, Ca, Al, Zn, Na and K containing glasses are preferred.

### Porosity Description

The substrate's porosity is another relevant aspect to producing a catalyst composition of the invention. Generally, the substrate should be substantially nonporous, though materially insignificant amounts of micro-, meso-and/or macro-pore volume may exist without adversely affecting the catalyst composition's intended use. Because micropore volume in a material is often difficult to detect, two surface area measurements are used herein to determine whether a substrate is substantially nonporous for identifying the catalyst composition of the invention.

The first surface area measurement, useful for detecting the extent of micro-, meso- and/or macro-porosity, is determined by a thermal adsorption/desorption method suitable for the expected surface area range being measured. For example, for higher surface area measurements (e.g., > about 3 m²/g) N₂ BET, according to the method described by ASTM D3663-03, (“S.A._{N2-BET}”), would likely be a preferred surface area measurement technique. While for lower surface area measurements (e.g., < about 3 m²/g) Kr BET, according to the method described by ASTM D4780-95, (“S.A._{Kr-BET}”), would likely be a preferred surface area measurement technique. The most preferred surface area measurement for detecting the extent of micro-, meso- and/or macro-porosity will be apparent to one skilled in the art of analyzing solid and semi-solid material surface areas. The second measurement is a sodium-chemisorption surface area (“S.A._{Na}”), which can be expressed as a change vs. time in NaOH titrant using the type of analytical method described by R. Iler in Chemistry of Silica, John Wiley & Sons (1979) at p. 203 and 353 and defined more specifically above under the S.A._{Na} rate of change (“SARC_{Na}”).

Accordingly, as defined herein, the substrate will be substantially nonporous, provided the substrate's S.A._{N2-BET} or S.A._{Kr-BET} is in a range from about 0.01 m²/g to about 10 m²/g and its SARC_{Na} is less than or equal to 0.5, which, as discussed more fully above, is the ratio of two volumes of NaOH titrant, wherein the denominator of the ratio is the volume of NaOH titrant solution used initially, to titrate at time zero, tₒ, a substrate slurry mixture containing 1.5 g of the substrate in 3.4M NaCl solution from pH 4 to pH 9 at about 25° C. But again, as noted above, before the initial NaOH titration begins for the SARC_{Na} determination, the aqueous slurry mixture must first be adjusted to pH 4, using either a small amount of acid (HCl) or base (NaOH), accordingly. Also, as explained above, the cumulative volume of NaOH titrant used at three 5-minute intervals, to maintain the substrate slurry mixture at pH 9 over 15 minutes is Vₜₒₜₐₗ-Vᵢ(i.e., V_{5 to 15}), the numerator of the ratio SARC_{NA}. So, if Vₜₒₜₐₗ-Vᵢ is less than or equal to 0.5Vᵢ, the corresponding SARC_{Na} is less than or equal to 0.5. Accordingly, a substrate with a SARC_{Na}≦0.5 will be substantially non-porous as defined herein, provided, again, that the substrate's S.A._{N2-BET} or S.A._{Kr-BET} is also in a range from about 0.01 m²/g to about 10 m²/g. Provided these surface area parameters are satisfied, to the extent the substrate has any micropore, mesopore and/or macropore volume, it would be an insufficient concentration, distribution and/or type to adversely affect the catalyst composition's expected performance for its intended use.

The sodium surface area (“S.A._{Na}”) is an empirical titration procedure developed for essentially pure forms of SiO₂ in the granular, powder, and suspended sol form. The S.A._{Na} is a measure of the reactivity and accessibility of surface protonic sites (Glass-O⁻H⁺), which for pure SiO₂ would correspond to Si—O⁻H⁺ sites. The behavior of silicate glasses and crystalline silicates, which markedly differ in composition from pure SiO₂ with respect to the stoichiometry of this titration procedure, is not known or predictable in terms of the absolute value of the NaOH titrant measured in the S.A._{Na} experiment. The equations used by Sears and Iler to correlate the NaOH volume of the S.A._{Na} experiment with the N₂-BET surface area of the SiO₂ materials studied, therefore, are not valid for reliably predicting the absolute surface areas of more complex silicate compositions. This is expected since the Glass-O⁻H⁺ groups that can be present in compositionally diverse glasses can include such moieties such as Al—O⁻H⁺, B—O⁻H⁺, Ti—O⁻H⁺, as well as more structurally diverse protonic groups associated with multiple Si—O⁻H⁺ moieties on a single Si site (Q² groups), etc. On the other hand, the total surface area of “silica-like” glass compositions, such as leached quartz, for example, might well be reliably determined using the S.A._{Na} experiment, provided the minimum pore size is in a range accessible to standard gas phase BET measurements, since it's comprised primarily of networked SiO₂ and Si—O⁻H⁺ moieties. However the diffusional accessibility of the Glass-O⁻H⁺ moieties to hydroxide ions (OH⁻) and sodium ions (Na⁺), and hence the relative percentage of microporous vs. mesoporous, macroporous and/or substantially nonporous regions, should be detectable based on the amount of NaOH that must be added (titrant) vs. time in the S.A._{Na} experiment to maintain the final pH of 9. So, in sum, the accessibility of Glass-O⁻H⁺ moieties to OH⁻ and Na⁺ versus time, as determined by the SARC_{Na} experiment described above, can be taken as a reasonably reliable measure of the presence of microporosity, including porosity of a type that may not be accessible to standard gas phase BET measurements.

Preferably, the substrate's surface area will remain substantially unchanged after its ion leach treatment, which is often the case with most alkali resistant (“AR”) glasses. However, in certain cases there may be some ion depletion from the substrate network without significantly affecting the substrate's micropore structure, if any, and thereby avoiding an adverse effect on the catalyst composition's expected performance for its intended use. But to the extent there is significant ion depletion and concomitant leaching from the substrate network, microporous regions in the substrate are likely created. Accordingly, as noted above, this microporous structure is indicated by a SARC_{Na} greater than about 0.5. A substrate network exhibiting these properties has developed sufficient micropore structure, particularly in the subsurface region, that would likely have an adverse effect on the substrate's capacity to sustain its surface active state, and hence, adversely affect the catalyst composition's expected performance for its intended use.

### Substrate Shapes, Forms and Size Description

Shapes and forms of the substrates used for producing the catalyst composition of the invention are diverse. Examples of suitable shapes include, without limitation, fibers, fibrillated fibers, cylindrical particles (e.g., pellets), spherical particles (e.g., spheres), elliptical particles (e.g., ellipsoids), flat particles (e.g., flakes), irregular fractured particles, spiral or helical particles and combinations thereof.

Examples of suitable formed bodies or composites that such substrate shapes can take include, without limitation, woven composites, nonwoven composites, mesh fabrics, extrudates, rings, saddles, cartridges, membranes, spiral bound membranes, filters, fiber tows, chopped fibers and combinations thereof.

In some instances, depending on the catalyst composition's intended use, the bodies or composites (collectively, “composites”) may be formed with a catalytic substrate using any one of a variety of suitable materials as forming media, including, without limitation, boehmite, hydrous titania and TiO₂, hydrous zirconia and ZrO₂, gamma alumina, alpha alumina, silica, clays, natural and synthetic polymeric fibers, polymeric resins, and solvent and water soluble polymers, whether the substrate contains Type-1 or Type-2 catalytic constituents (described more fully below). Preferably, the catalytic substrate should be positioned so that it's located on or substantially near the outer surface of the composite (i.e., on the outer periphery of the composite). Without being bound by theory, it's believed that placement of a substantial portion of the catalytic substrate on and/or in the outer peripheral region of the catalyst composite (“composite periphery”) will reduce the extent to which undesired intra-composite diffusional effects could be introduced.

So, it should be understood that a suitable distance for positioning a substantial portion of the catalytic substrate in and/or on the composite periphery will depend on the catalyst composite's intended use, the catalyst composite's overall dimensions and shape and the catalytic substrate's overall dimensions and shape. Accordingly, over a diversity of composite shapes and sizes, the mean thickness of this composite periphery, in and/or on which catalytic substrates can be placed, will generally range from about 1 micron to about 400 microns. Preferably, however, the mean thickness of this composite periphery ranges from about 1 micron to about 250 microns and more preferably from about 1 micron to about 150 microns.

Depending on the catalyst composition's intended use, however, there may be instances where distributing the substrate substantially throughout the forming media may be desirable. For example, without limitation, in processes where extended exposure of the reactants and/or reaction intermediates is desirable, it may be preferable to composite the substrate (again, whether Type-1 or Type-2 catalytically active substrate) substantially throughout the forming media, preferably, though not necessarily, having a controlled pore size distribution.

The minimum size of the substrates (i.e., substrate particle's mean maximum dimension) used for producing the formed bodies or composites are generally in a range from greater than about 0.05 microns to less than or equal to about 150 microns, preferably from about 0.2 microns to less than or equal to about 150 microns and more preferably from about 0.2 microns to about 50 microns. However, substrates outside this range could still be effective, for instance in continuous fiber forms given above, without adversely affecting the catalyst composition's expected performance, depending on the composition's intended use and other process variables potentially affected by catalyst composition's shape and form.

It will be understood by those skilled in the art that the compositing operation will likely introduce potential macro-, meso-, and/or micro-porosity into the finished composite. This porosity is, however, not introduced into the functionalized surface component of the catalyst composition, as described herein, during the compositing operation.

### II. Substrate Surface Activation

Substrates used for producing the catalyst composition of the invention can be made surface active with one or more first constituents having a first type of ionic and/or electrostatic interaction with the substrate (“Type-1 constituent precursor”). As more fully explained below, a Type-1 constituent precursor may itself be catalytically effective or may be further treated to produce a catalytically active region, having a mean thickness ≦ about 30 nm, preferably, ≦ about 20 nm and more preferably, ≦ about 10 nm, on and/or in the substrate surface. For example, in certain instances, depending on the catalyst compositions intended use, provided the substrate obtained has the appropriate type and degree of pore structure (if any) and an isoelectric point (IEP) in the range suitable for the intended use, the substrate may be sufficiently surface active, as received, to be catalytically effective. Optionally, though preferably, the substrates can be treated to further modify and/or enhance their surface activity. Also, optionally, the substrates can be treated to remove any organic coatings or other possible contaminants that would be expected to interfere with the catalyst composition's performance. Also, as discussed more fully below, under “Type-2 Constituent Precursor Integration Treatment,” depending on the catalyst composition's intended use, it may be preferable, to further treat the substrate's surface with an ion exchange (IEX), back ion exchange (BIX) and/or electrostatic adsorption (EA) treatment method that integrates one or more second constituents on and/or in the substrate surface having a second type of ionic and/or electrostatic interaction with the substrate, which produces a catalytically active region, having a mean thickness ≦30 nm, preferably, ≦20 nm and more preferably, ≦10 nm, on and/or in the substrate surface, accordingly.

### Substrate Contaminant Removal Treatment

A contaminant removal treatment may be optional depending on the composition of the substances typically found on the surface of the substrate and whether such substances would be expected to interfere with catalyst composition's preparation and/or its expected performance for the intended use. For example, AR-glass is typically manufactured with an organic coating (i.e., sizing) used to facilitate its processing, such as dispersion in aqueous formulations. This organic coating or sizing, however, may interfere with the catalyst composition's preparation, if not its catalytic performance for at least most, if not, all intended uses. Accordingly, the organic coating should be removed.

Calcination is a preferred method for removing such an organic coating. Because the primary objective of this treatment is contaminant removal from the substrate, the conditions for this type of calcination treatment are not particularly crucial to the substrate's successful surface activation. In certain instances, depending on the nature of the contaminant to be removed from the substrate a solvent, surfactant, aqueous wash or other suitable means can be used to satisfactorily remove the contaminant.

To the extent calcination is used, however, it's preferable to calcine the substrate in an oxidizing atmosphere (e.g., under air or O₂). Also, it's important to select a calcination temperature high enough to remove the targeted contaminants, but low enough to reasonably avoid the material's softening point. Generally, the calcination temperature should be at least about 50° C. below the selected substrate material's softening point. Preferably, the calcination temperature should be at least about 100° C. below the selected substrate material's softening point. In the case of AR-glass, for example, an acceptable contaminant removal calcination temperature can range from about 300° C. to about 700° C. for most AR-glass types. Generally, the selected substrate material should be calcined for about 2 to 14 hours and preferably about 4 to 8 hours. Nonetheless, this calcination time can vary beyond these times, depending on the nature of the substrate obtained and the contaminants targeted for removal from the substrate.

### Surface Activation by Ion-Leach Treatment

After any potential contaminants are substantially removed from the substrate, the substrate can then be treated to produce a surface active state and a desired isoelectric point (“IEP”), provided the initial IEP obtained with the substrate is not in the desired range. In some cases, however, a substrate, as-received, may be sufficiently surface active to be further modified by one or more of the other treatments described more fully below, without a first-type ion-leach (IEX-1) treatment, first discussed in more detail among the other treatments described more fully below. In other words, the elemental composition of the substrate, particularly at or substantially near the external surface, may be sufficient to obtain the desired IEP. In many cases, however, the substrate's elemental composition will require some modification to shift its initial IEP and obtain an IEP suitable, in turn, for the desired surface active state, in type and degree, depending on the catalyst composition's intended use.

This surface active state, with one or more first constituents having (i) a first oxidation state and (ii) a first type of ionic and/or electrostatic interaction with the substrate may be sufficient for producing a catalytically active region, having a mean thickness ≦ about 30 nm, preferably, ≦ about 20 nm and more preferably, ≦ about 10 nm, on and/or in the substrate surface, and accordingly, providing the catalyst composition's expected performance for the intended use. For example, without limitation, Bronsted or Lewis acid sites and Bronsted or Lewis base sites on and/or in the substrate's surface can be effective for promoting some hydrocarbon, hetero-hydrocarbon (e.g., oxygen containing hydrocarbon) and non-hydrocarbon treatment, conversion and/or refining processes.

In other instances, however, based on the catalyst composition's intended use, it may be preferable to further treat the substrate surface with one or more of the ion exchange methods described below for (i) a second oxidation state, which can be the same or different from that of the first oxidation state and (ii) a second type of ionic and/or electrostatic interaction with the substrate sufficient for producing a catalytically active region, having a mean thickness ≦30 nm, preferably, ≦20 nm and more preferably, ≦10 nm, on and/or in the substrate surface.

Turning now to the surface activation treatment, the treatment involves at least one ion-leaching treatment to obtain a first type or Type-1 ion exchanged (IEX-1) substrate. It should be understood, however, that where the substrate, as-received, has as a suitable IEP for the catalyst composition's intended use, IEX-1 is also intended to describe this first type of substrate.

Generally, this ion-leaching treatment is performed by any suitable method effective for removing the desired ionic species in a substantially heterogeneous manner across the substrate surface without significantly eroding the substrate network (e.g., avoiding production of any micropore structure either in the surface region and/or subsurface region). For example, without limitation, most acids, whether inorganic or organic, and various chelating agents are suitable for use in the ion-leaching treatment. Preferably, inorganic acids are used, for example, without limitation, nitric acid, phosphoric acid, sulfuric acid, hydrochloric acid, acetic acid, perchloric acid, hydrobromic acid, chlorosulfonic acid, trifluoroacetic acid and combinations thereof.

Generally, the strength of an acid solution used in an ion-leaching treatment depends on the properties of the substrate (e.g., affinity of ion(s) to be removed from the glass network, strength of the glass after certain network ions are removed, etc.), the extent to which the substrate's IEP needs to be shifted and the catalyst composition's intended use. Preferably, the strength of an acid solution used in an ion-leaching treatment can range from about 0.5 wt. % to about 50 wt. %, more preferably ranges from about 2.5 wt. % to about 25 wt. % and most preferably ranges from about 5 wt. % to about 10 wt. %.

Chelating agents may also be used in an ion-leaching treatment. For example, without limitation, ethylenediaminetetraacetic acid (“EDTA”), crown ethers, oxalate salts, polyamines, polycarboxylic acids and combinations thereof.

Generally, the strength of a chelating agent solution used in an ion-leaching treatment depends on the properties of the substrate (e.g., affinity of ion(s) to be removed from the glass network, strength of the glass after certain network ions are removed, etc.) and the catalyst composition's intended use. Preferably, the strength of an chelating agent solution used in an ion-leaching treatment can range from about 0.001 wt. % to saturation, more preferably ranges from about 0.01 wt. % to saturation.

Generally, heat treatment conditions, such as heating temperature, heating time and mixing conditions, for the ion-leaching treatment are selected in view of the type and strength of the acid or chelating agent used and the properties of the substrate.

Depending on the strength of the acid or chelating agent solution, the heating temperature can be widely varied. Preferably, however, the heating temperature for an acidic, ion-leaching treatment ranges from about 20° C. to about 200° C. and more preferably from about 40° C. to about 95° C. and most preferably from about 60° C. to about 90° C. Preferably, the heating temperature for chelating, ion-leaching treatment ranges range from about 20° C. to about 200° C. and more preferably from about 40° C. to about 90° C.

Depending on the strength of the acid or chelating agent solution and the heating time, the heating time for the ion-leaching treatment can be varied. Preferably, the heating time for the ion-leaching treatment ranges from about 15 minutes to about 48 hours, more preferably ranges from about 30 minutes to about 12 hours.

Generally, mixing conditions are selected in view of the type and strength of the acid or chelating agent used and the properties of the substrate (e.g., affinity of ion(s) to be removed from the glass network, strength of the glass after certain network ions are removed, etc.) and the duration of the heat treatment. For example, without limitation, mixing conditions may be continuous or intermittent, and may be mechanical, fluidized, tumbling, rolling, or by hand.

In sum, the combination of acid or chelating strength, heat treatment conditions and mixing conditions are determined in view of obtaining a sufficient degree of ion-exchange (“IEX”) between the acid or chelating agent and the targeted substrate ion(s) necessary for producing a suitable isoelectric point and type and degree of surface charge needed to produce the surface active state desired for either the substrate's subsequent treatment(s) or the catalyst composition's intended use.

After the ion-leaching treatment is completed the ion-leach treated substrate is preferably isolated by any suitable means, including, without limitation, filtration means, centrifuging means, decanting and combinations thereof. Thereafter, the ion-leach treated substrate is washed with one or more suitable rinsing liquid(s), such as deionized water and/or suitable water-soluble organic solvent (e.g., methanol, ethanol or acetone) and dried at about room temperature to 110° C. for about 20 to 24 hours.

### Back-Ion Exchange Treatment

In some instances, depending on the catalyst composition's intended use, it may be preferable to subject the selected substrate to a back-ion exchange (“BIX”), or two-step ion exchange treatment, collectively referred to herein as a BIX treatment. A BIX treatment is described as a “back-ion” exchange, without limitation, generally because ions of one type (e.g., Na⁺) that are removed from the substrate with an ion-leach treatment are subsequently put back into or returned to the substrate by mixing the ion-leached substrate with a salt solution (e.g., NaCl) comprising ions of the type initially removed. Whether the ions that are removed from the substrate are necessarily returned to the same site they initially occupied in the substrate is not clear. But regardless of whether the initially displaced ions are site-shifted, in whole, in part or not at all, from the BIX treatment, it should be understood that the BIX treatment described herein covers all catalyst compositions arising from any of these possible ion-site placement variations.

Generally, the types of salt solutions used for treating an ion-leach treated substrate will depend on the type of ion(s) to be back-ion exchanged. Preferably, only one type of ion will be back-ion exchanged, but it may be desirable in certain instances to back-ion exchange two or more ions.

Any ions susceptible to removal using the ion-leaching treatment described above can be back-ion exchanged. Some examples of such ions include, without limitation, ions of alkali metals from Group 1 (formerly Group IA), such as Li, Na and K, and alkaline earth metals from Group 2 (formerly Group IIA), such as Be, Mg, Ca, NH₄⁺ and alkylammonium cations, and small organic polycations. Preferably, alkali metal ions and NH₄⁺ are preferred target ions for a BIX treatment, while Na⁺ and NH₄⁺ are preferred BIX ions and Na⁺ is a particularly preferred BIX ion.

Generally, the concentration of the salt solutions used for the BIX treatment will depend on the type of ion-leach treated substrate undergoing a BIX treatment and the BIX ion's relative affinity for returning to the ion-leach treated substrate, again, regardless of the site the BIX-ion returns to in the substrate network (e.g., Na⁺ relative affinity for the substrate vs. H⁺). For most types of glass substrates, such as, without limitation, AR, A or quartz glass, about a 0.001 mol/L to 5 mol/L strength BIX-salt solution is preferred, while about a 0.05 mol/L to 3 mol/L BIX-salt solution is more preferred.

Typically, heat treatment conditions, such as heating temperature, heating time and mixing conditions, for the BIX treatment are selected in view of the type and strength of the BIX-salt solution used and the properties of the substrate.

Preferably, the heating temperature for BIX treatment using BIX-salt solution can range from about 20° C. to about 200° C. and more preferably from about 30° C. to about 95° C.

Depending on the strength of the BIX-salt solution and the heating temperature selected, the heating time for the BIX treatment can be varied. Preferably, the heating time for the BIX treatment ranges from about 5 minutes to about 24 hours, more preferably ranges from about 30 minutes to about 8 hours.

Generally, mixing conditions are selected in view of the type and strength of the BIX salt solution used and the properties of the substrate (e.g., affinity of ion(s) to be removed from the glass network, strength of the glass after certain network ions are removed, etc.) and the duration of the heat treatment. For example, without limitation, mixing conditions may be continuous or intermittent, and may be mechanical, fluidized, tumbling, rolling or by hand.

In sum, the combination of BIX salt solution strength, heat treatment conditions and mixing conditions are based substantially on returning a sufficient amount and distribution of BIX-ion back to the substrate, regardless of its siting in the substrate network, necessary for producing the type and degree of surface charge needed to produce the surface active state desired for either the substrate's subsequent treatment(s) or the catalyst composition's intended use.

### Adjusting Substrate Surface Charge by pH Adjustment

Preferably, a negative surface charge on the substrate is desired to sustain an electrostatic interaction or affinity with a positively charged constituent(s) (e.g., cationic alkali earth metal, a cationic transition metal constituent, etc.). However, for some potential catalyst composition uses, a positive surface charge may be desirable to support an electrostatic interaction or affinity with a negatively charged constituent (e.g., an anionic transition metal oxyanion, sulfate anion, noble metal polyhalide anion, etc.).

As a general rule, the surface charge of the substrate can be shifted to either a net positive or net negative state by adjusting the pH of an ion-leach treated substrate/IEX mixture either below or above the substrate's isoelectric point (“IEP”). Recall, the IEP is also known as zero point charge (“ZPC”). So, put another way the IEP (or ZPC) can be viewed as the pH at which the surface of a material at incipient wetness has a net zero surface charge. So, adjusting the pH of a substrate/IEX water mixture to a pH greater than the substrate's IEP (or ZPC) produces a net negative surface charge on the substrate. Alternatively, adjusting the pH of a substrate/IEX water mixture to a pH less than the substrate's IEP (or ZPC) produces a net positive surface charge on the substrate.

For example, without limitation, where an AR-glass has an IEP equal to 9.6, adjusting the pH of an ion-leach treated AR-glass to a pH>9.6 will produce a net negative surface charge on the surface of the glass. Depending on the IEP profile of the AR-glass, it may be preferable to adjust the pH by one or perhaps two or more pH units above the glass substrate's IEP to ensure its surface charge is well sustained.

The types of solutions used for making such a pH adjustment will depend on compatibility with other reagents, glass stability and desired charge density, among other factors. Generally any dilute base can be used to adjust the substrate's surface charge to the right of its IEP (i.e., to produce net negative surface charge) and any dilute acid can be used to adjust the substrate's surface charge to the left of its IEP (i.e., to produce net positive surface charge). Either inorganic or organic acids and bases can be used in a dilute strength, with inorganic acids generally being preferred. Generally the strength of the dilute acid or base solution will depend on the type of acid or base used and its dissociation constant and the pH suitable for obtaining the desired type and density of surface charge.

In some cases it may be desirable to integrate a catalytic constituent or precursor at a pH that produces a surface charge of the same sign as the ionic catalytic constituent or precursor. Under these conditions, the electrostatic adsorption (EA) type mechanism of integration is not probable. However, without being bound by theory, direct ion exchange (IEX) or back exchange (BIX) at exchangeable surface sites can occur, resulting in a surface integration of the catalytic constituent or precursor that is possibly physically and/or chemically different from the same component integrated under the electrostatic adsorption (EA) mechanism. For instance, certain substrate surface moieties containing a cation (or anion) susceptible to displacement by an ionic catalytic constituent or precursor of the same sign can provide the exchange sites for discreet, but nonetheless effective, IEX or BIX with the substrate's surface moieties. For example, without limitation, moieties such as, siloxy (—Si—O⁻Na⁺) moieties contain Na⁺ ions that can be displaced, at least in part, by a positively charged catalytic metal or metal complex precursor, such as, without limitation, Pd(NH₃)₄²⁺, to produce a substrate with a catalytically effective amount of catalytic constituents.

### pH Adjustment to Control Surface Charge of BIX Treated Substrate

As in the case of the IEX treatment or a second IEX treatment (“IEX-2 treatment”, discussed below), a pH adjustment may also be desired for certain BIX treatments, though not necessarily required. Again, the extent of pH adjustment required will depend generally on the substrate's IEP, its IEP vs. surface charge profile curve and the type of charge desired, in view of a second constituent to be integrated with the surface in an IEX-2 treatment, as well as the type of BIX-ion(s) exchanged.

The types of solutions used for making such a pH adjustment will depend on compatibility with other reagents, substrate stability in the pH range of interest and desired charge density, among other factors. Generally any dilute base can be used to adjust the substrate's surface charge to the right of its IEP (i.e., to produce net negative surface charge) and any dilute acid can be used to adjust the substrate's surface charge to the left of its IEP (i.e., to produce net positive surface charge). Either inorganic or organic acids and bases can be used in a dilute strength. Generally the strength of the dilute acid or base solution will depend on the type of acid or base used and its dissociation constant and a pH suitable for obtaining the desired type and density of surface charge.

### III. Type-2 Constituent Precursor Integration Treatment

Whether the substrate is surface active, as received, or is an ion-leach treated substrate (i.e., IEX-1 treated substrate), or BIX-treated substrate, preferably, the substrate is further treated with at least one second constituent precursor (“Type-2 constituent precursor”) in either (i) a second ion exchange (“IEX-2”) treatment, (ii) an electrostatic adsorption (EA) treatment or (iii) some combination of an IEX-2 and EA treatment, for integrating one or more second constituent precursors on and/or in the substrate surface having a second type of ionic and/or electrostatic interaction with the substrate. In turn, depending on the intended use, some Type-2 constituent precursors, without further treatment, can produce a catalytically active region or, subject to further treatment, can produce a catalytically active region comprising one or more Type-2 constituents. But whether the catalytically active region is comprised of (a) a Type-2 constituent precursor, (b) a Type-2 constituent, arising from Type-2 constituent precursor(s), or (c) some combination thereof, the catalytic region has a mean thickness ≦ about 30 nm, preferably, ≦ about 20 nm and more preferably, ≦ about 10 nm on and/or in the substrate surface.

As noted previously, in some instances, an as received substrate or ion-leach treated substrate can be catalytically effective depending on the catalyst composition's intended use. However, for many potential uses, it will often be preferable to subject the selected substrate to an IEX-2 and/or EA treatment. For example, without limitation, the reaction rate, selectivity and/or energy efficiency of many processes suitable for using the catalyst compositions of the invention can be significantly enhanced by displacing at least a portion of the first constituent (“Type-1 constituent”) and integrating a second type of constituent (“Type-2 constituent”) with the substrate surface.

Without being bound by theory, Type-2 constituent precursor ions can be integrated by direct or indirect ionic interaction with oppositely charged specific ion exchange sites on and/or in the substrate surface, by electrostatic adsorption interaction with an oppositely charged substrate surface, some combination thereof or some other type of precursor-charge-to-surface interaction, yet to be understood. But regardless of the nature of the interaction the Type-2 constituent precursor(s) may have with an as-received substrate, IEX-1 treated, or BIX-treated substrate, a second type of precursor charge-to-surface interaction is produced that will, accordingly, produce a catalytically active region, having a mean thickness ≦ about 30 nm, preferably, ≦ about 20 nm and more preferably, ≦ about 10 nm, on and/or in the substrate surface.

Strictly for ease of discussion below and without intending to limit the scope of the invention described herein, IEX-2 will be used herein to collectively refer to the diverse range of interactions generally described as Type-2 constituent precursor charge-to-surface interaction or Type-2 constituent precursor interactions.

Generally, the types of salt solutions used for treating an IEX-1 treated or BIX-treated substrate will depend on the type of ion(s) to be ion exchanged in the IEX-2 treatment. Either one type of ion will be ion exchanged, or it may be desirable in certain instances to ion exchange two or more ions, either concurrently or sequentially.

In the case where two different types of constituent precursor ions are integrated with substrate, the IEX-2 treatment is referred to herein as a double ion-exchange or double IEX-2 treatment. Accordingly, where three different types of constituent precursor ions are integrated with substrate, the IEX-2 treatment is called a triple ion-exchange or triple IEX-2 treatment.

### Type-2 Constituent and Precursor Description

Any salt solutions of IEX-2 ions chemically susceptible to either displacing ions on the as-received, IEX-1 treated, or BIX-treated substrate surface or having a charge affinity for electrostatically interacting with IEX-1 treated or BIX-treated substrate surface can be used.

So, IEX-2 ions are precursors to constituents that can be used as Type-2 constituents. As noted above, depending on their intended use, these ionic IEX-2 precursors (i.e., Type-2 constituent precursors) may be catalytically effective and, if so, can work, in their precursor state, like Type-2 constituents in one type of catalyst composition, even though such ions can also work as IEX-2 precursors in the preparation of another type of catalyst composition. Generally, however, ionic IEX-2 precursors (useful for obtaining Type-2 constituents integrated with the substrate surface), include, without limitation, Bronsted or Lewis acids, Bronsted or Lewis bases, noble metal cations and noble metal complex cations and anions, transition metal cations and transition metal complex cations and anions, transition metal oxyanions, transition metal chalconide anions, main group oxyanions, halides, rare earth ions, rare earth complex cations and anions and combinations thereof.

Again, depending on the catalyst composition's intended use, certain IEX-2 ions can themselves be catalytically effective in the precursor state, when integrated with the appropriate substrate, to produce a Type-2 constituent. Some examples of ionic IEX-2 precursors that, optionally, without further treatment, can be catalytically effective include, without limitation, Bronsted or Lewis acids, Bronsted or Lewis bases, noble metal cations, transition metal cations, transition metal oxy anions, main group oxyanions, halides, rare earth hydroxides, rare earth oxides, and combinations thereof.

Some examples of noble and transition metals useful as precursors to Type-2 constituents include, without limitation, ionic salts and complex ion salts of Groups 7 through 11 (formerly Groups Ib, IIb, Vb, VIb, Vb, VIII), such as Pt, Pd, Ni, Cu, Ag, Au, Rh, Ir, Ru, Re, Os, Co, Fe, Mn, Zn and combinations thereof. Ionic salts of Pd, Pt, Rh, Ir, Ru, Re, Cu, Ag, Au, and Ni are particularly preferred for an IEX-2 treatment. For convenience, the elements of these groups may be seen, for example, in a Periodic Table of the Elements presented at http://pearl1.Ianl.gov/periodic/default.htm using the IUPAC system of numbering the groups (as well as presenting formerly used group numbers).

Some examples of transition metal oxyanions useful as Type-2 constituent precursors include, without limitation, ionic salts of Group 5 and 6 (formerly Groups Vb and VIb), such as VO₄³⁻, WO₄²⁻, H₂W₁₂O₄₀⁶⁻.MoO₄²⁻, Mo₇O₂₄⁶⁻, Nb₆O₁₉⁶⁻, ReO₄⁻, and combinations thereof. Ionic salts of Re, Mo, W and V are particularly preferred for an IEX-2 treatment.

Some examples of transition metal chalconide anions useful as Type-2 constituent precursors include, without limitation, ionic salts of Group 6 (formerly Group VIb), such as MoS₄²⁻, WS₄²⁻, and combinations thereof.

Some examples of main group oxyanions useful as Type-2 constituent precursors include, without limitation, ionic salts of Group 16 (formerly Group VIa), such as SO₄²⁻, PO₄³⁻, SeO₄²⁻, and combinations thereof. Ionic salts of SO₄²⁻ are particularly preferred for an IEX-2 treatment.

Some examples of halides useful as Type-2 constituent precursors include, without limitation, ionic salts of Group 17 (formerly Group VIIa), such as F⁻, Cl⁻, Br⁻, I⁻ and combinations thereof. Ionic salts of F⁻ and Cl⁻ are particularly preferred for an IEX-2 treatment.

Some examples of rare earth ions and rare earth complex cations or ions useful as Type-2 constituent precursors include, without limitation, ionic salts of the lanthanides and actinides, such as La, Pr, Nd, Pm, Sm, Eu, Gd, Tb, Dy, Ho, Er, Tm, Yb, Lu, Th, U, and combinations thereof.

Some examples of transition metals that can be used to produce transition metal-carbides, -nitrides, -borides, and -phosphides useful as Type-2 constituents include, without limitation, ionic salts of Cr, Mo, W, Nb, Ta, Fe, Co, Ni, and combinations thereof.

### IEX-2 Treatment Description

Generally, the concentration of the salt solutions used for the IEX-2 treatment will depend on the type of IEX-1 treated or BIX-treated substrate undergoing a IEX-2 treatment and the IEX-2 ion's relative affinity for interacting and/or integrating with the IEX-1 treated substrate. For most types of glass substrates, such as, without limitation, AR, A or soda-lime glass, about a 0.001 wt. % to saturation of the IEX-2 salt solution is preferred, while about a 0.001 wt. % to 5 wt. % IEX-2 salt solution is more preferred. However, depending on the functional surface concentration of catalytic constituent(s) considered necessary for the catalyst composition's intended use, IEX-2 salt solutions may be less than 0.001 wt. %.

Where multiple ion types are exchanged with the substrate, whether concurrently or sequentially, the concentration of salt solutions will be adjusted according to the relative loading desired for each type of constituent precursor on the substrate and the substrate's relative affinity for one type of constituent precursor vs. another. For example, without limitation, in a double IEX-2 treatment (i.e., two different catalytic constituent precursor types integrated with the IEX-1 or BIX-treated substrate) or triple IEX-2 treatment (i.e., three different catalytic constituent precursor types integrated with the IEX-1 or BIX-treated substrate) the concentration of the salt solutions used for depositing each ion type will depend on the relative concentration targeted for each type of constituent precursor integrated with the substrate's surface and the surface's affinity for each ion.

Typically, heat treatment conditions, such as heating temperature, heating time and mixing conditions, for the IEX-2 treatment are selected in view of the type and strength of the IEX-2 salt solution used and the properties of the substrate.

Preferably, the heating temperature for IEX-2 treatment using an acid can range from about 20° C. to about 200° C. and more preferably from about 30° C. to about 90° C.

Depending on the strength of the IEX-2 salt solution and the heating temperature selected, the heating time for the IEX-2 treatment can be varied. Preferably, the heating time for the IEX-2 treatment ranges from about 5 minutes to about 48 hours, more preferably ranges from about 30 minutes to about 5 hours.

Generally, mixing conditions are selected in view of the type and strength of the IEX-2 salt solution used and the properties of the substrate (e.g., affinity of ion(s) to be removed from the glass network, strength of the glass after certain network ions are removed, etc.) and the duration of the heat treatment. For example, without limitation, mixing conditions may be continuous or intermittent, and may be mechanical, fluidized, tumbling, rolling, or by hand.

In sum, the combination of IEX-2 salt solution strength, heat treatment conditions and mixing conditions are based substantially on integrating a sufficient amount and distribution of IEX-2 ions on and/or in the substrate, regardless of the nature of its physicochemical association with the substrate's surface, necessary for producing the type and degree of surface charge needed to produce the surface active state desired for the catalyst composition's intended use.

### Adjusting Substrate Surface Charge by pH Adjustment

As discussed previously, the extent of pH adjustment required will depend generally on the substrate's IEP, its IEP vs. surface charge profile curve and the type of charge desired, in view of Type-2 constituent precursor(s) to be integrated with the surface in a second IEX (“IEX-2”) treatment. For example, without limitation, for a substrate with an IEP of 8, the pH of the substrate/IEX-2 mixture is preferably adjusted to within a range from about 8 to about 12 and more preferably, from about 9 to about 11.

The types of solutions used for making such a pH adjustment will depend on compatibility with other reagents, substrate stability in the pH range of interest and desired charge density, among other factors. Generally any dilute base can be used to adjust the substrate's surface charge to the right of its IEP (i.e., to produce net negative surface charge) and any dilute acid can be used to adjust the substrate's surface charge to the left of its IEP (i.e., to produce net positive surface charge). Either inorganic or organic acids and bases can be used in a dilute strength, with organic bases generally being preferred. Generally the strength of the dilute acid or base solution will depend on the type of acid or base used, its dissociation constant, and pH suitable for obtaining the desired type and density of surface charge.

After the IEX-2 treatment is completed the IEX-2 treated substrate is preferably isolated by any suitable means, including, without limitation, filtration means, centrifuging means, decanting and combinations thereof. Thereafter, the IEX-2 treated substrate is washed with one or more suitable rinsing liquid(s), such as distilled or deionized water, dilute base or acid and/or suitable water-soluble organic solvent (e.g., methanol, ethanol or acetone) and dried at about 110° C. for about 20 to 24 hours.

### IV. Post-Deposition Treatment Description

Optionally, after the IEX-2 treated substrate is isolated it may be dried, calcined only, calcined under oxidizing conditions and subsequently reduced or further oxidized, reduced without calcination or oxidized without calcination. Reaction of surface deposited transition metal ions, oxyanions and/or thioanions in the gas or liquid phase with suitable reducing, sulfiding, carbiding, nitriding, phosphiding, or boriding reagents (-IDING reagents) can be carried out as desired to produce the respective catalytically effective metal sulfide/oxysulfide, metal carbide/oxycarbide, metal nitride/oxynitride, metal boride, or metal phosphide constituent.

Generally, without limitation, the purpose of the post-deposition calcination treatment is to substantially decompose the metal counterion or ligands and more intimately integrate the metal, metal oxide, metal chalconide, and the like with the substrate surface and remove any residual water that may not have been removed from the previous drying treatment.

The conditions for such a calcination treatment for an IEX-2 treated substrate are not particularly crucial to the substrate's successful surface activation, however, they should only be severe enough to produce at least one catalytically-active region with the deposited constituent precursor(s) in a catalytically effective amount. But to the extent calcination is used, the substrate is first calcined in an oxidizing atmosphere (e.g., under air or O₂). Also, it's important to select a calcination temperature high enough to ensure the Type-2 constituent precursor of interest is oxidized and any residual water removed (if any is still present), but low enough to reasonably avoid the substrate's softening point and undesired decomposition of the deposited constituent precursor(s).

For example, without limitation, deposited sulfate requires calcination conditions to decompose associated cations and anchor the sulfate to the surface but the conditions must not significantly decompose the sulfate to volatile sulfur oxides. Similarly, metal oxyanions require calcination conditions that decompose the associated cations and anchor the anion to the surface as an oxide, but the conditions must not be severe enough to volatilize the metal oxide from the surface or cause the metal oxide to dissolve into the substrate. Finally, noble metals and complexes should be calcined under conditions that decompose the ligands and anions present, but not severe enough to agglomerate the noble metal on the surface. For this reason, preferably, noble metals are directly reduced, without calcination, as described more fully below.

Generally, the calcination temperature should be at least about 100° C. below the selected substrate substrate's softening point. The calcination temperature should be from about 100° C. to 700° C., preferable from about 200° C. to 600° C., and most preferably from about 300° C. to 500° C.

Typically, the IEX-2 treated substrate is calcined for about 1 to about 24 hours and preferably about 2 to about 12 hours. Nonetheless, this calcination time can vary beyond these times, depending on the Type-2 constituent integrated with the substrate.

Generally, without limitation, the purpose of the post-deposition reducing treatment is to, at least substantially, if not fully, reduce catalytic constituent precursors such as metals, metal oxides or metal sulfides to a lower oxidation state integrated with the substrate surface. Examples of suitable reducing agents include, without limitation, CO and H₂. H₂ is a preferred reducing agent, preferably at a flow rate in a range from about 0.01 L/hr. to about 100 L/hr. per gram of substrate, and more preferably at a flow rate of about 0.1 L/hr. to 1 L/hr. per gram of substrate.

Typically, the reducing temperature should be about 0° C. to 600° C., provided the chosen temperature is at least 100° C. below the softening point of the substrate.

Generally, the IEX-2 treated substrate undergoes a reducing treatment for about 0.1 to about 48 hours and preferably about 1 to about 8 hours.

Alternatively, the IEX-2 treated substrate may be reduced by a solution phase treatment with a soluble reducing agent such as, without limitation, hydrazine, sodium hydride, lithium aluminum hydride and combinations thereof in a suitable solvent such as water or an ether.

Generally, without limitation, the purpose of the post deposition -IDING reaction treatment simultaneously reduces the metal ions, metal oxyanions, and/or metal thioanions while additionally reacting the reduced metal with a lower atomic weight -IDING element-containing reagent. In certain cases direct -IDING takes place without simultaneous reduction of the metal oxidation state, for instance in certain sulf-IDING treatments.

Typical gas phase -IDING reagents include, without limitation, hydrogen sulfide, methyl mercaptan and dimethylsulfide (sulf-IDING reagents), ammonia (nitr-IDING reagent), methane, ethane, and other light hydrocarbons (carb-IDING reagents). These gas-phase -IDING reagents can be reacted directly or in a gas blend with an inert gas or hydrogen at ambient or elevated pressure with an IEX-2 treated substrate to produce the corresponding sulfide, carbide or nitride. Partially -IDED species, including oxysulfides, oxycarbides, and oxynitrides, which may be catalytically effective, can also be produced by incomplete reaction with either substrates in a substantially as-received/obtained condition, integrated IEX-2 treated substrates, calcined IEX-2 treated substrates, or reduced IEX-2 treated substrates.

Metal phosphides can be made by reducing treatment of doubly ion exchanged (double IEX-2 treatment) substrates wherein one of the IEX-2 treatments is one or more transition metal ions and the other IEX-2 treatment is phosphate ion. Preferably, the two IEX-2 treatments can be carried out sequentially. Also, metal phosphides can be made by using gas-phase phosph-IDING reagent for example, without limitation, phosphine (PH₃), to produce the desired metal phosphide. For example, a single ion exchanged substrate (single IEX-2 treated substrate) with the desired transition metal in the suitable oxidation state can be further treated with PH₃ to produce the desired metal phosphide, accordingly.

Solution phase treatments can be used to produce metal sulfide, metal boride, and metal phosphide catalytic constituents. Typical solution treatments that produce metal sulfides include, without limitation, treatment of IEX-2 treated metal-ion-integrated substrate with effective concentrations of organic solutions of hexamethyldisilthiane from room temperature to reflux temperature for a time sufficient to yield a catalytically effective amount of catalytic constituent on and/or in the substrate surface.

Typical solution phase treatments that produce borides include, without limitation, aqueous sodium borohydride or potassium borohydride treatment of IEX-2 treated metal-ion-integrated substrate at temperatures from room temperature to reflux for an effective time. Typical solution phase treatments that produce phosphides include aqueous sodium hypophosphite treatment of IEX-2 treated metal-ion-integrated substrate at temperatures from room temperature to reflux for a time sufficient to yield a catalytically effective amount of catalytic constituent on and/or in the substrate surface.

### V. Catalytically-Active Region Description

The catalytically-active region arising from any of the above-described substrate treatments, will have (i) a mean thickness less than or equal to about 30 nm, preferably, ≦ about 20 nm and more preferably, ≦ about 10 nm and (ii) a catalytically effective amount of at least one type of catalytic constituent. The mean thickness of the catalytic region is preferably determined using XPS spectroscopy using a technique of layer-by-layer etching known as sputter depth profiling (discussed more fully under the Analytic Methods in the Examples provided below). However, other analytical techniques known to those skilled in the art may be used to determine the general locus of a catalytic constituent versus the surface of the constituent's related substrate. So, the mean thickness of a substrate's catalytic region may be determined for example, without limitation, using transmission electron microscopy (TEM) or scanning TEM (STEM, also described more fully below). The XPS or TEM procedures are each well understood by those skilled in the art.

It should be understood that, in the limit, the thickness of a catalytically-active region, whether arising from an IEX-1 treatment or IEX-2 treatment (with or without a BIX treatment), will not, on average, (a) penetrate substantially beyond the substrate's surface region or (b) exceed about a 30 nm thickness, preferably, about a 20 nm thickness and more preferably, about a 10 nm thickness, above the substrate's external surface, for any catalyst composition of the invention. Regarding the positioning of one or more catalytically-active regions on and/or in a treated substrate, it should also be understood that the catalytically-active region(s) may be:
(a) on the substrate's external surface and, to the extent any pores are present, on the substrate's pore wall surface;
(b) in the substrate's surface region, that is, about 30 nm beneath, preferably, about 20 nm beneath and more preferably, about 10 nm beneath, the substrate's external surface and, to the extent any pores are present, about 30 nm beneath, preferably, about 20 nm beneath and more preferably, about 10 nm beneath, the substrate's pore wall surface, but above the substrate's subsurface region, accordingly;
(c) on or above the substrate's external surface and, to the extent any pores are present, on or above the substrate's pore wall surface, in part, and located in the substrate's surface region, in part, or
(d) combinations of (a), (b) and (c).

Generally, amounts of catalytic constituents, whether Type-1 constituents or Type-2 constituents, can range from about 0.0002 wt. % to about 5 wt. %, preferably from about 0.0002 wt. % to about 2 wt. % and more preferably from about 0.0005 wt. % to about 1 wt. %. Furthermore, the catalytically-active region(s) of the catalyst compositions of the invention may be contiguous or discontiguous.

Without being bound by theory, it is believed that catalyst compositions with discontiguous coverage of catalytically-active regions are at least equally, and in some cases, more effective, than catalyst composition's with substantially contiguous or more extensive areas of contiguous coverage of catalytically-active regions. The extent of the catalytically-active region's external surface coverage on the substrate can range from as low as about 0.0001% coverage to as high as 100% coverage. Preferably, the extent of the catalytically-active region's external surface coverage ranges from about 0.0001% to about 10% and more preferably from about 0.0001% to about 1%. But again, without being bound by theory, it's generally believed that catalyst composition's, particularly those with lower wt. % loadings of catalytic constituents, will likely be more catalytically effective as the catalytically-active regions on and/or in the treated substrate become more highly dispersed (i.e., a greater degree of distribution and separation between catalytically-active regions).

The catalytically-active region and other catalyst composition attributes described above are based on the inventors' best available information about the catalyst composition's state before entering a steady-state reaction condition. The extent to which one or more of the described attributes may change is uncertain and in large measure unpredictable. Nonetheless, without being bound by theory, it's believed that the functional surface active nature of the catalyst compositions described herein will allow, among other composition attributes, the charge and/or geometric orientation of the catalytic constituents integrated with the substrate to vary significantly as a catalyst composition facilitates its intended process reaction. Accordingly, it should be understood that the scope of the invention described herein extends as well to all catalyst compositions arising from the claimed compositions placed under a steady state reaction condition.

### VI. Catalyst Composition Applications in Hydrogenation Processes

Generally, it's expected that the catalyst compositions of the type described above will be most beneficial to processes where the activity or selectivity of the catalyst is limited by intraparticle diffusional resistance of product or reactants (i.e., diffusion limited processes). However, the catalyst compositions can also be used on processes that are not necessarily diffusion limited. For example, without limitation, some processes may simply require the unique type catalytic interaction offered by the type of catalyst compositions described above to help lower a particular process reaction's activation energy. In turn, a lower activation energy can make the process more thermodynamically favorable (e.g., less energy required to drive the process), and hence, more cost effective to perform on a commercial scale.

Non-selective hydrogenation (or for brevity, simply “hydrogenation”) processes are one class of processes in which the catalyst compositions described above can be beneficially used to treat hydrocarbons, hetero-hydrocarbons and mixtures thereof. Hydrocarbons, as used herein, means a group of chemical compounds composed only of carbon (C) and hydrogen (H) atoms, while hetero-hydrocarbons, as used herein, means a group of chemical compounds composed primarily of C and H, but also contain at least one other atom other than C and H, such as, for example, without limitation, oxygen (O), nitrogen (N) and/or sulfur (S).

In a hydrogenation process, process streams with hydrocarbons and/or hetero-hydrocarbons suitable for hydrogenation using the catalyst compositions of the type described above generally include hydrocarbons with 1 to about 30 carbon atoms and perhaps one or more heteroatoms (e.g., O, N, S, etc.), but in certain instances, can have more than 30 carbon atoms, wherein the hydrocarbon has at least one hydrogenatable site that is susceptible to hydrogenation under suitable hydrogenation conditions (described more fully below) for the desired product, yield and/or process efficiency.

A process stream includes, without limitation, a feed stream, an intermediate transfer stream, a recycle stream and/or discharge stream. As used herein, a hydrogenatable site means a site having at least one atom that's either a carbon (C) atom or a heteroatom, but generally is a carbon-containing site, while the heteroatom may be, for example, without limitation, oxygen (O), nitrogen (N) or sulfur (S). In either case, however, a hydrogenatable site has at least one degree of unsaturation and is susceptible to being at least partially saturated in the presence of a catalyst composition under appropriate reaction conditions.

Also, the degree and type of unsaturated sites in the hydrocarbon may vary. So, for example, polyenes, polyynes and cyclenes may have a double bond and/or triple bond sites that are successive (successive double-double bonds only), conjugated or separated by one or more saturated and/or substituted carbons. Process streams suitable for hydrogenation can also have mixtures of alkenes or polyenes, aromatics or cyclenes, alkynes or polyynes and/or hetero-hydrocarbons having at least one hydrogenatable site.

Accordingly, hydrocarbons and/or hetero-hydrocarbons suitable for hydrogenation using catalyst compositions of the type described above include, without limitation, alkenes, dienes, polyenes, alkynes, polyynes, cyclenes, aromatics, unsaturated vegetable oils and hydrogenatable oxygenates. Hydrogenatable oxygenates include for example, without limitation, ketones, aldehydes, carboxylic acids, quinones and other hetero-hydrocarbons having one or more heteroatoms other than O, such as, N or S, for example.

A preferred group of hydrocarbons suitable for hydrogenation using the catalyst compositions of the type described above is the group of normal olefins, normal polyenes and normal alkynes with 2 to about 20 carbon atoms and aromatics with 6 to 12 carbon atoms, whether substituted or unsubstituted. More preferred hydrocarbons are normal olefins, normal polyenes, olefin-substituted aromatics, normal alkynes, olefinic-aldehydes and olefinic-ketones having 2 to 15 carbon atoms and aromatics with 6 to 8 carbon atoms.

Generally, a hydrogenation process can be carried out using a various types of reactors having one or more hydrogenation zones so that the reactant hydrocarbon feed stream are sufficiently contacted with the catalyst composition in a hydrogenation zone maintained under hydrogenation conditions (described more fully below). This contacting can be performed in a fixed catalyst bed system, a moving catalyst bed system, a fluidized bed system, or in a batch-type operation using a variety of catalyst composites, such as those described above.

Generally, a fixed bed system is preferred. In a fixed bed system the hydrocarbon feed stream is preheated to the desired reaction temperature and then flowed into the hydrogenation zone containing a fixed bed of the catalyst composite. The hydrogenation zone may itself comprise one or more separate reaction zones, with heating means therebetween, to ensure that the desired reaction temperature is maintained on the input-side of each reaction zone. The hydrocarbon may be contacted with the catalyst bed in an upward, downward or radial flow fashion. Radial flow of the hydrocarbon through the catalyst bed is preferred. The hydrocarbon may be in the liquid phase, a mixed vapor-liquid phase or the vapor phase when it contacts the catalyst. Preferably, it is in the vapor phase.

Hydrogenation conditions under which the catalyst compositions described above can be used in many hydrogenation processes, again, depending on the desired product, yield and/or process efficiency, include (a) temperatures generally ranging from about 0° C. to about 538° C., (b) pressures generally ranging from about 101 kPa to about 13,789 kPa, (c) mole ratios of H₂ to hydrogenatable hydrocarbon generally ranging from about 0.1:1 to about 20:1 and (d) liquid hourly space velocities (LHSV) in the reactor generally ranging from about 0.1 hr⁻¹ to about 20 hr⁻¹.

### EXAMPLES

The present invention is described in further detail in connection with the following examples which illustrate or simulate various aspects involved in the practice of the invention. It is to be understood that all changes that come within the spirit of the invention are desired to be protected and thus the invention is not to be construed as limited by these examples.

### Catalyst Composition with Alkali Resistant (AR) Glass Substrate

### Example 1

### Palladium on AR-Glass

AR-glass Cem-FIL Anti-Crak™ HD, sample, as glass fibers having a mean diameter of about 17-20 microns, produced by Saint-Gobain Vetrotex, is obtained.

First, the as-received AR-glass sample undergoes a calcination heat treatment. In that treatment, the AR-glass is calcined at 600° C. for 4 hrs in air under an air flow rate of 1 L/hr.

Second, the calcined AR-glass undergoes an acid-leach treatment. 25 g of the calcined AR-glass and 3 L 5.5 wt. % nitric acid are each placed in a 4-L wide-neck plastic container. The plastic container is placed in an air draft oven at 60° C. for 1 hr and shaken briefly by hand every 15 minutes. After the acid-leach treatment is completed, the sample is filtered on a Buchner funnel with Whatman 541 paper and washed with about 7.6 L deionized water. Thereafter, the acid-leached sample is dried at 110° C. for 22 hrs.

Third, the acid-leach treated AR-glass undergoes an ion-exchange (IEX) treatment. In this example, palladium tetraamine-dihydroxide, [Pd(NH₃)₄](OH)₂, is used to prepare 80 mL 0.1 wt. % palladium solution for ion exchange (“IEX solution”). 4 g of AR-glass is added to the IEX solution (“glass/IEX mixture”). The pH of the glass/IEX mixture is measured, resulting in a pH of about 11.4. The mixture is then transferred to a 150-mL wide neck plastic container. The container is placed in an air-draft oven at 50° C. for 2 hrs and shaken briefly by hand every 30 minutes. After the IEX treatment is completed, the glass/IEX mixture is filtered on a Buchner funnel with Whatman 541 paper and washed with about 3.8 L deionized water. Thereafter, the IEX-glass is dried at 110° C. for 22 hrs.

Fourth, the IEX-glass undergoes a reducing treatment in which the IEX-glass is initially calcined at 300° C. for 2 hrs in air at an air flow rate of 2 L/hr and thereafter reduced at 300° C. for 4 hrs in hydrogen (H₂) under a H₂ flow rate of 2 L/hr.

The sample is analyzed by Inductively Coupled Plasma-Atomic Emission Spectroscopy (ICP-AES), resulting in a palladium concentration of about 0.016 wt. %.

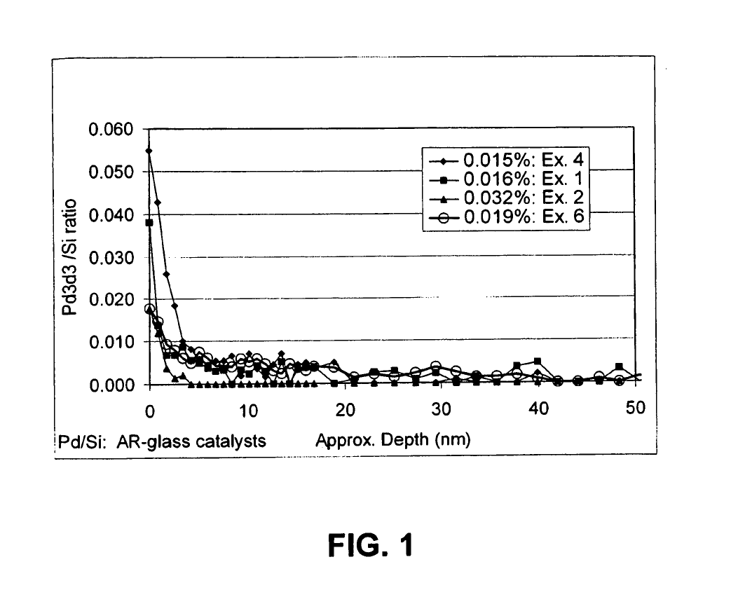

The sample is analyzed by an XPS Sputter Depth Profiling method (as described below), demonstrating, as depicted in FIG. 1, that the thickness of the region in which a substantial portion of the palladium is detected by this method is about 10 nm.

### Example 2

### Palladium on AR-Glass

AR-glass Cem-FIL Anti-Crak™ HD, sample, as glass fibers having a mean diameter of about 17-20 microns, produced by Saint-Gobain Vetrotex, is obtained and prepared according to the procedure of Example 1.

The sample is analyzed by ICP-AES, resulting in a palladium concentration of about 0.032 wt. %.

The sample is analyzed by an XPS Sputter Depth Profiling method (as described below), demonstrating, as depicted in FIG. 1, that the thickness of the region in which a substantial portion of the palladium is detected by this method is about 10 nm.

### Example 3

### Palladium on AR-Glass

AR-glass Cem-FIL Anti-Crak™ HD, sample, as glass fibers having a mean diameter of about 17-20 microns, produced by Saint-Gobain Vetrotex, is obtained.

First, the as-received AR-glass sample undergoes a calcination heat treatment. In that treatment, the AR-glass is calcined at 600° C. for 4 hrs in air under an air flow rate of 1 L/hr.

Second, the calcined AR-glass undergoes an acid-leach treatment. 25 g of the calcined AR-glass and 3 L 5.5 wt. % nitric acid are each placed in a 4-L wide-neck plastic container. The plastic container is placed in an air draft oven at 60° C. for 1 hr and shaken briefly by hand every 15 minutes. After the acid-leach treatment is completed, the sample is filtered on a Buchner funnel with Whatman 541 paper and washed with about 7.6 L deionized water. Thereafter, the acid-leached sample is dried at 110° C. for 22 hrs.

Third, the acid-leach treated AR-glass undergoes an IEX treatment. In this example, palladium tetramine-dichloride, [Pd(NH₃)₄](Cl)₂, is used to prepare 40 mL 0.1 wt. % palladium solution for IEX (“IEX solution”). 4 g of AR-glass is added to the IEX solution (“glass/IEX mixture”). The pH of the glass/IEX mixture is measured, resulting in a pH of about 7.7. The mixture is then transferred to a 100-mL wide neck plastic container and placed in an air-draft oven at 50° C. for 2 hrs and shaken briefly by hand every 30 minutes. After the IEX treatment is completed, the glass/IEX mixture is filtered on a Buchner funnel with Whatman 541 paper and washed with about 3.8 L deionized water. Thereafter, the IEX-glass sample is dried at 110° C. for 22 hrs.

Fourth, the IEX-glass sample undergoes a reducing treatment in which the IEX-glass is initially calcined at 300° C. for 2 hrs in air at an air flow rate of 2 L/hr and thereafter reduced at 300° C. for 4 hrs in hydrogen (H₂) under a H₂ flow rate of 2 L/hr.

The sample is analyzed by ICP-AES, resulting in a palladium concentration of about 0.0012 wt. %.

### Example 4

### Palladium on AR-Glass

AR-glass Cem-FIL Anti-Crak™ HD, sample, as glass fibers having a mean diameter of about 17-20 microns, produced by Saint-Gobain Vetrotex, is obtained.

First, the as-received AR-glass sample undergoes a calcination heat treatment. In that treatment, the AR-glass is calcined at 600° C. for 4 hrs in air under an air flow rate of 1 L/hr.

Second, the calcined AR-glass undergoes an acid-leach treatment. About 50 g of the calcined AR-glass and 4 L 5.5 wt. % nitric acid are each placed in a 4-L wide-neck plastic container. The plastic container is put into an air draft oven at 90° C. for 2 hr and shaken briefly by hand every 30 minutes. After the acid-leach treatment is completed, the sample is filtered on a Buchner funnel with Whatman 541 paper and washed with about 7.6 L deionized water. Thereafter, the acid-leached sample is dried at 110° C. for 22 hrs.

Third, the acid-leached AR-glass undergoes a Na⁺-back-ion exchange (“Na—BIX”) treatment. The acid-leached sample from the second step is mixed with 4 L 3 mol/L sodium chloride (NaCl) solution (“glass/NaCl mixture”). The pH of the glass/NaCl mixture is measured. As needed, the pH of the mixture is adjusted with a continuous drop-wise addition of about 40 wt. % tetrapropylammonium-hydroxide to greater than pH 10 (in this example, resulting in a pH of about 11.0). The glass/NaCl mixture is transferred to a 4-L wide neck plastic container. The container is subsequently placed in an air-draft oven at 50° C. for 4 hrs and shaken briefly by hand every 30 minutes. After the Na—BIX treatment is completed, the glass/NaCl mixture is filtered and the Na—BIX/AR-glass sample collected on a Buchner funnel with Whatman 541 paper and washed with about 7.6 L deionized water. Thereafter, the Na—BIX/AR-glass sample is dried at 110° C. for 22 hrs.

Fourth, Na—BIX/AR-glass sample undergoes a second ion-exchange (“IEX-2”) treatment. In this example, palladium tetramine-chloride, [Pd(NH₃)₄](Cl)₂, is used to prepare 3 L 0.01 wt. % palladium solution for ion exchange (“IEX-2 solution”). 42 g of Na—BIX/AR-glass is added to the IEX-2 solution (“glass/IEX-2 mixture”). The pH of the glass/IEX-2 mixture is measured, resulting in a pH of about 8.5. The mixture is then transferred to a 4-L wide neck plastic container. The container is placed in an air-draft oven at 100° C. for 22 hrs and shaken briefly by hand several times over the 22 hr heating period. After the IEX-2 treatment is completed, the glass/IEX-2 mixture is filtered and the IEX-2-glass sample collected on a Buchner funnel with Whatman 541 paper is washed with about 7.6 L of a dilute ammonium hydroxide (NH₄OH) solution. The dilute NH₄OH solution is prepared by mixing 10 g of a concentrated 29.8 wt. % NH₄OH solution with about 3.8 L of deionized water. Thereafter, the IEX-2-glass sample is dried at 110° C. for 22 hrs.

Fifth, the IEX-2-glass sample undergoes a reducing treatment in which the sample is reduced at 300° C. for 4 hrs in hydrogen (H₂) under a H₂ flow rate of 2 L/hr.

The sample is analyzed by ICP-AES, resulting in a palladium concentration of about 0.015 wt. %.

The sample is analyzed by an XPS Sputter Depth Profiling method (as described below), demonstrating, as depicted in FIG. 1, that the thickness of the region in which a substantial portion of the palladium is detected by this method is about 10 nm.

### Example 5

### Palladium on AR-Glass

AR-glass Cem-FIL Anti-Crak™ HD, sample, as glass fibers having a mean diameter of about 17-20 microns, produced by Saint-Gobain Vetrotex, is obtained.

First, the as-received AR-glass sample undergoes a calcination heat treatment. In that treatment, the AR-glass is calcined at 600° C. for 4 hrs in air under an air flow rate of 1 L/hr.

Second, the calcined AR-glass undergoes an acid-leach treatment. 90.03 g of the calcined AR-glass and 4 L 5.5 wt. % nitric acid are each placed in a 4-L wide-neck plastic container. The plastic container is placed in an air draft oven at 90° C. for 2 hr and shaken briefly by hand every 15 minutes. After the acid-leach treatment is completed, the sample is filtered on a Buchner funnel with Whatman 541 paper and washed with about 7.6 L deionized water. Thereafter, the acid-leached sample is dried at 110° C. for 22 hrs.

Third, the acid-leach treated AR-glass undergoes an ion-exchange (IEX) treatment. In this example, palladium tetraamine-dihydroxide, [Pd(NH₃)₄](OH)₂, is used to prepare 2000 mL 0.1 wt. % palladium solution for ion exchange (“IEX solution”). 80.06 g of AR-glass is added to the IEX solution (“glass/IEX mixture”). The pH of the glass/IEX mixture is measured, resulting in a pH of about 10.6. The mixture is then transferred to a 4000-mL wide neck plastic container. The container is placed in an air-draft oven at 50° C. for 72 hrs and shaken briefly by hand every 30 minutes. After the IEX treatment is completed, the glass/IEX mixture is filtered on a Buchner funnel with Whatman 541 paper and washed with about 7.6 L a dilute NH₄OH solution. The dilute NH₄OH solution is prepared by mixing 10 g of a concentrated 29.8 wt. % NH₄OH solution with about 3.8 L of deionized water. Thereafter, the IEX-glass sample is dried at 110° C. for 22 hrs.

Fourth, the IEX-glass undergoes a reducing treatment in which the IEX-glass is reduced at 300° C. for 4 hrs in hydrogen (H₂) under a H₂ flow rate of 2 L/hr.

The sample is analyzed by ICP-AES, resulting in a palladium concentration of about 0.019 wt. %.

### Example 6

### Palladium on AR-Glass

AR-glass Cem-FIL Anti-Crak™ HD, sample, as glass fibers having a mean diameter of about 17-20 microns, produced by Saint-Gobain Vetrotex, is obtained.

First, the as-received AR-glass sample undergoes a calcination heat treatment. In that treatment, the AR-glass is calcined at 600° C. for 4 hrs in air under an air flow rate of 1 L/hr.

Second, the calcined AR-glass undergoes an acid-leach treatment. 250 g of the calcined AR-glass and 3 L 5.5 wt. % nitric acid are each placed in a 1-L wide-neck glass container. The open plastic container is heated for 2 hrs on a Corning hotplate to a temperature of 90-100° C. on the bottom of the container an to at least 75° C. at the top of the container, measured with thermocouples placed at several places in the container; 5.5 wt. % nitric acid is added to keep the volume at 3 L as solution evaporates during the treatment. After the acid-leach treatment is completed, the sample is filtered on 200 mesh stainless steel screen and washed with about 15 L deionized water. Thereafter, the acid-leached sample is dried at 100° C. for several hours.

Third, the acid-leach treated AR-glass undergoes an ion-exchange (IEX) treatment. In this example, palladium tetraamine-dihydroxide, [Pd(NH₃)₄](OH)₂, is used to prepare 2000 mL 0.1 wt. % palladium solution for ion exchange (“IEX solution”). 80 g of AR-glass is added to the IEX solution (“glass/IEX mixture”). The pH of the glass/IEX mixture is measured, resulting in a pH of about 9.4. The mixture is then transferred to a 4000-mL wide neck plastic container. The container is placed in an air-draft oven at 50° C. for 2 hrs and shaken briefly by hand every 30 minutes. After the IEX treatment is completed, the glass/IEX mixture is filtered on a Buchner funnel with Whatman 541 paper and washed with about several liters of deionized water. Thereafter, the IEX-glass is dried at 110° C. for 22 hrs.

Fourth, the IEX-glass undergoes a reducing at 300° C. for 4 hrs in hydrogen (H₂) under a H₂ flow rate of 2 L/hr.

The sample is analyzed by ICP-AES, resulting in a palladium concentration of about 0.019 wt. %.

The sample is analyzed by an XPS Sputter Depth Profiling method (as described below), demonstrating, as depicted in FIG. 1, that the thickness of the region in which a substantial portion of the palladium is detected by this method is about 10 nm.

### Example 7

### Platinum on AR-Glass

AR-glass Cem-FIL Anti-Crak™ HD, sample, as glass fibers having a mean diameter of about 17-20 microns, produced by Saint-Gobain Vetrotex, is obtained.

First, the as-received, AR-glass sample undergoes a calcination heat treatment. In that treatment, the AR-glass is calcined at 600° C. for 4 hrs in air under an air flow rate of 1 L/hr.

Second, the calcined AR-glass undergoes an acid-leach treatment. About 160 g of the calcined AR-glass and 12 L 5.5 wt. % nitric acid are each placed in a 15-L round bottom flask and stirred mechanically with a stainless steel paddle stirrer at 300-500 rpm while heated at 90° C. for 2 hrs. After the acid-leach treatment is completed, the sample is filtered on a Buchner funnel with Whatman 541 paper and washed with about 7.5 L deionized water. Thereafter, the acid-leached sample is dried at 110° C. for 22 hrs. The acid-leached sample is subsequently milled to a fine powder using by a single pass through a small-scale hammer mill.

Third, the milled, acid-leach treated AR-glass undergoes an IEX treatment. In this example, platinum tetraamine-dichloride, [Pt(NH₃)₄](Cl)₂, is used to prepare 1 L 0.3 wt. % platinum solution for ion exchange (“IEX solution”). About 158 g of the milled, acid-leach treated AR-glass is added to the IEX solution (“glass/IEX mixture”). The pH of the glass/IEX mixture is measured. As needed, the pH of the mixture is adjusted with a continuous drop-wise addition of about 29.8 wt. % ammonium hydroxide (NH₄OH) to greater than pH 10 (in this example, resulting in a pH of about 10.6). The glass/IEX mixture is transferred to a 4-L beaker and heated at 50° C. for 2 hrs with continuous mechanical stirring with a stainless steel paddle stirrer at 300-500 rpm. After 1 hr of heating the pH is again measured, and as needed, adjusted again with about 29.8 wt. % NH₄OH solution to a pH greater than 10. At the completion of the 2 hr. heating period, the glass/IEX mixture's pH is again measured, resulting in a pH of about 10.1. After the IEX treatment is completed, the glass/IEX mixture is filtered and IEX-glass sample collected on a Buchner funnel with Whatman 541 paper and washed with about 7.6 L of a dilute NH₄OH solution. The dilute NH₄OH solution is prepared by mixing 10 g of a concentrated 29.8 wt. % NH₄OH solution with about 3.8 L of deionized water. Thereafter, the IEX-glass sample is dried at 110° C. for 22 hrs.

Fourth, the IEX-glass sample undergoes a reducing treatment in which the ion-exchanged sample is reduced at 300° C. for 4 hrs in hydrogen (H₂) under a H₂ flow rate of 2 L/hr.

The sample is analyzed by ICP-AES, resulting in a platinum concentration of about 0.0033 wt. %.

### Example 8

### Platinum on AR-Glass

AR-glass Cem-FIL Anti-Crak™ HD, sample, as glass fibers having a mean diameter of about 17-20 microns, produced by Saint-Gobain Vetrotex, is obtained.

First, the as-received, AR-glass sample undergoes a calcination heat treatment. In that treatment, the AR-glass is calcined at 600° C. for 4 hrs in air under an air flow rate of 1 L/hr.

Second, the calcined AR-glass undergoes an acid-leach treatment. About 30 g of the calcined AR-glass and 4 L 5.5 wt. % nitric acid are each placed in a 4-L wide neck plastic container. The plastic container is placed in an air draft oven at 90° C. oven for 2 hrs and shaken briefly by hand every 30 minutes. After the acid-leach treatment is completed, the sample is filtered on a Buchner funnel with Whatman 541 paper and washed with about 7.5 L deionized water. Thereafter, the acid-leached sample is dried at 110° C. for 22 hrs.

Third, acid-leach treated AR-glass undergoes an IEX treatment. In this example, platinum tetraamine-dichloride, [Pt(NH₃)₄](Cl)₂, is used to prepare 3 L 0.01 wt. % platinum solution for ion exchange (“IEX solution”). About 15.01 g of acid-leach treated AR-glass is added to the IEX solution (“glass/IEX mixture”). The pH of the glass/IEX mixture is measured. As needed, the pH of the mixture is adjusted with a continuous drop-wise addition of about 29.8 wt. % ammonium hydroxide (NH₄OH) to greater than pH 10 (in this example, resulting in a pH of about 10.6). The glass/IEX mixture is transferred to a 4-L wide neck plastic container. The plastic container is placed in an air draft oven at 50° C. oven for 2 hrs and shaken briefly by hand every 30 minutes. After 1 hr of heating the pH is again measured, and as needed, adjusted again with about 29.8 wt. % NH₄OH solution to a pH greater than 10. At the completion of the 2 hr. heating period, the glass/IEX mixture's pH is again measured, resulting in a pH of about 10.19. After the IEX treatment is completed, the glass/IEX mixture is filtered and IEX-glass sample collected on a Buchner funnel with Whatman 541 paper and washed with about 7.6 L of a dilute NH₄OH solution. The dilute NH₄OH solution is prepared by mixing 10 g of a concentrated 29.8 wt. % NH₄OH solution with about 3.8 L of deionized water. Thereafter, the IEX-glass sample is dried at 110° C. for 22 hrs.

Fourth, the IEX-glass undergoes a reducing treatment in which the IEX-glass is reduced at 300° C. for 4 hrs in hydrogen (H₂) under a H₂ flow rate of 2 L/hr.

The sample is analyzed by ICP-AES, resulting in a platinum concentration of about 0.0032 wt. %.

### Example 9

### Platinum on AR-Glass

AR-glass Cem-FIL Anti-Crak™ HD, sample, as glass fibers having a mean diameter of about 17-20 microns, produced by Saint-Gobain Vetrotex, is obtained.

First, the as-received, AR-glass sample undergoes a calcination heat treatment. In that treatment, the AR-glass is calcined at 600° C. for 4 hrs in air under an air flow rate of 1 L/hr.

Second, the calcined AR-glass undergoes an acid-leach treatment. About 30 g of the calcined AR-glass and 4 L 5.5 wt. % nitric acid are each placed in a 4-L wide neck plastic container. The plastic container is placed in an air draft oven at 90° C. oven for 2 hrs and shaken briefly by hand every 30 minutes. After the acid-leach treatment is completed, the sample is filtered on a Buchner funnel with Whatman 541 paper and washed with about 7.5 L deionized water. Thereafter, the acid-leached sample is dried at 110° C. for 22 hrs.

Third, acid-leach treated AR-glass undergoes an IEX treatment. In this example, platinum tetraamine-dichloride, [Pt(NH₃)₄](Cl)₂, is used to prepare 3 L 0.01 wt. % platinum solution for ion exchange (“IEX solution”). About 9.8 g of acid-leach treated AR-glass is added to the IEX solution (“glass/IEX mixture”). The pH of the glass/IEX mixture is measured. As needed, the pH of the mixture is adjusted with a continuous drop-wise addition of about 40 wt. % tetrapropylammonium-hydroxide to greater than pH 10 (in this example, resulting in a pH of about 11.38). The glass/IEX mixture is transferred to a 4-L wide neck plastic container. The plastic container is placed in an air draft oven at 100° C. oven for 22 hrs and shaken briefly by hand every 30 minutes. After the IEX treatment is completed, the glass/IEX mixture is filtered and IEX-glass sample collected on a Buchner funnel with Whatman 541 paper and washed with about 7.6 L of a dilute NH₄OH solution. The dilute NH₄OH solution is prepared by mixing 10 g of a concentrated 29.8 wt. % NH₄OH solution with about 3.8 L of deionized water. Thereafter, the IEX-glass sample is dried at 110° C. for 22 hrs.

Fourth, the IEX-glass undergoes a reducing treatment in which the IEX-glass is reduced at 300° C. for 4 hrs in hydrogen (H₂) under a H₂ flow rate of 2 L/hr.

The sample is analyzed by ICP-AES, resulting in a platinum concentration of about 0.038 wt. %.

### Example 10

### Platinum on AR-Glass

AR-glass Cem-FIL Anti-Crak™ HD, sample, as glass fibers having a mean diameter of about 17-20 microns, produced by Saint-Gobain Vetrotex, is obtained.

First, the as-received, AR-glass sample undergoes a calcination heat treatment. In that treatment, the AR-glass is calcined at 600° C. for 4 hrs in air under an air flow rate of 1 L/hr.

Second, the calcined AR-glass undergoes an acid-leach treatment. About 30 g of the calcined AR-glass and 4 L 5.5 wt. % nitric acid are each placed in a 4-L wide neck plastic container. The plastic container is placed in an air draft oven at 90° C. oven for 2 hrs and shaken briefly by hand every 30 minutes. After the acid-leach treatment is completed, the sample is filtered on a Buchner funnel with Whatman 541 paper and washed with about 7.5 L deionized water. Thereafter, the acid-leached sample is dried at 110° C. for 22 hrs.

Third, acid-leach treated AR-glass undergoes an IEX treatment. In this example, platinum tetraamine-dichloride, [Pt(NH₃)₄](Cl)₂, is used to prepare 3 L 0.01 wt. % platinum solution for ion exchange (“IEX solution”). About 8.79 g of acid-leach treated AR-glass is added to the IEX solution (“glass/IEX mixture”). The pH of the glass/IEX mixture is measured. As needed, the pH of the mixture is adjusted with a continuous drop-wise addition of about 29.8 wt. % ammonium hydroxide (NH₄OH) to greater than pH 10 (in this example, resulting in a pH of about 10.4). The glass/IEX mixture is transferred to a 4-L wide neck plastic container. The plastic container is placed in an air draft oven at 100° C. oven for 22 hrs and shaken briefly by hand every 30 minutes. After the IEX treatment is completed, the glass/IEX mixture is filtered and IEX-glass sample collected on a Buchner funnel with Whatman 541 paper and washed with about 7.6 L of a dilute NH₄OH solution. The dilute NH₄OH solution is prepared by mixing 10 g of a concentrated 29.8 wt. % NH₄OH solution with about 3.8 L of deionized water. Thereafter, the IEX-glass sample is dried at 110° C. for 22 hrs.

Fourth, the IEX-glass undergoes a reducing treatment in which the IEX-glass is reduced at 300° C. for 4 hrs in hydrogen (H₂) under a H₂ flow rate of 2 L/hr.

The sample is analyzed by ICP-AES, resulting in a platinum concentration of about 0.022 wt. %.

### Example 11

### Cobalt on AR-Glass

AR-glass Cem-FIL Anti-Crak™ HD, sample, as glass fibers having a mean diameter of about 17-20 microns, produced by Saint-Gobain Vetrotex, is obtained.

First, the as-received, AR-glass sample undergoes a calcination heat treatment. In that treatment, the AR-glass is calcined at 600° C. for 4 hrs in air under an air flow rate of 1 L/hr.

Second, the calcined AR-glass undergoes an acid-leach treatment. About 30 g of the calcined AR-glass and 4 L 5.5 wt. % nitric acid are each placed in a 4-L wide neck plastic container. The plastic container is placed in an air draft oven at 90° C. oven for 2 hrs and shaken briefly by hand every 30 minutes. After the acid-leach treatment is completed, the sample is filtered on a Buchner funnel with Whatman 541 paper and washed with about 7.5 L deionized water. Thereafter, the acid-leached sample is dried at 110° C. for 22 hrs.

Third, acid-leach treated AR-glass undergoes an IEX treatment. In this example, cobalt (II) nitrate hexahydrate, Co(NO₃)₂.6H₂O, is used to prepare 1 L 0.1 wt. % cobalt solution for ion exchange (“IEX solution”). The IEX solution is prepared by bubbling N₂ through 1 L of deionized water in an Erlenmeyer flask for 30 minutes to minimize the amount of air present to prevent cobalt from changing oxidation states upon addition. Then cobalt nitrate hexahydrate is added to the N₂-purged deionized water. The pH of the IEX solution is measured. As needed, the pH of the mixture is adjusted with a continuous drop-wise addition of about 29.8 wt. % ammonium hydroxide (NH₄OH) to greater than pH 10 (in this example, resulting in a pH of about 10.2). The IEX solution is transferred to a 1-L wide neck plastic container. About 20 g of acid-leach treated AR-glass is added to the IEX solution (“glass/IEX mixture”). The plastic container is placed in an air draft oven at 50° C. oven for 2 hrs and shaken briefly by hand every 30 minutes. After the IEX treatment is completed, the glass/IEX mixture is filtered on a Buchner funnel with Whatman 541 paper. The mother liquor is collected and pH measured (in this example pH is about 9.70). The filtered glass is then washed with about 6 L of a dilute NH₄OH solution. The dilute NH₄OH solution is prepared by mixing 10 g of a concentrated 29.8 wt. % NH₄OH solution with about 3.8 L of deionized water. Thereafter, the IEX-glass sample is dried at 110° C. for 16 hrs.

The sample is analyzed by ICP-AES, resulting in a cobalt concentration of about 0.64 wt. %.

### Example 12

### Cobalt on AR-Glass

AR-glass Cem-FIL Anti-Crak™ HD, sample, as glass fibers having a mean diameter of about 17-20 microns, produced by Saint-Gobain Vetrotex, is obtained.

First, the as-received, AR-glass sample undergoes a calcination heat treatment. In that treatment, the AR-glass is calcined at 600° C. for 4 hrs in air under an air flow rate of 1 L/hr.

Second, the calcined AR-glass undergoes an acid-leach treatment. About 30 g of the calcined AR-glass and 4 L 5.5 wt. % nitric acid are each placed in a 4-L wide neck plastic container. The plastic container is placed in an air draft oven at 90° C. oven for 2 hrs and shaken briefly by hand every 30 minutes. After the acid-leach treatment is completed, the sample is filtered on a Buchner funnel with Whatman 541 paper and washed with about 7.5 L deionized water. Thereafter, the acid-leached sample is dried at 110° C. for 22 hrs.

Third, acid-leach treated AR-glass undergoes an IEX treatment. In this example, cobalt (II) nitrate hexahydrate, Co(NO₃)₂.6H₂O, is used to prepare 1 L 0.1 wt. % cobalt solution for ion exchange (“IEX solution”). The IEX solution is prepared by bubbling N₂ through 1 L of deionized water in an Erlenmeyer flask for 30 minutes to minimize the amount of air present to prevent cobalt from changing oxidation states upon addition. Then cobalt nitrate hexahydrate is added to the N₂-purged deionized water. The pH of the IEX solution is measured. As needed, the pH of the mixture is adjusted with a continuous drop-wise addition of about 29.8 wt. % ammonium hydroxide (NH₄OH) to greater than pH 10 (in this example, resulting in a pH of about 10.24). The IEX solution is transferred to a 1-L wide neck plastic container. About 20 g of acid-leach treated AR-glass is added to the IEX solution (“glass/IEX mixture”). The plastic container is placed in an air draft oven at 50° C. oven for 45 minutes, shaken briefly by hand after 25 minutes. After the completion of the IEX treatment, the glass/IEX mixture is filtered on a Buchner funnel with Whatman 541 paper. The mother liquor is collected and pH measured (in this example pH is about 9.88). The filtered glass is then washed with about 6 L of a dilute NH₄OH solution. The dilute NH₄OH solution is prepared by mixing 10 g of a concentrated 29.8 wt. % NH₄OH solution with about 3.8 L of deionized water. Thereafter, the IEX-glass sample is dried at 110° C. for 17 hrs.

The sample is analyzed by ICP-AES, resulting in a cobalt concentration of about 0.15 wt. %.

### Example 13

### Tungsten on AR-Glass

AR-glass Cem-FIL Anti-Crak™ HD, sample, as glass fibers having a mean diameter of about 17-20 microns, produced by Saint-Gobain Vetrotex, is obtained.

First, the as-received, AR-glass sample undergoes a calcination heat treatment. In that treatment, the AR-glass is calcined at 600° C. for 4 hrs in air under an air flow rate of 1 L/hr.

Second, the calcined AR-glass undergoes an acid-leach treatment. About 30 g of the calcined AR-glass and 4 L 5.5 wt. % nitric acid are each placed in a 4-L wide neck plastic container. The plastic container is placed in an air draft oven at 90° C. oven for 2 hrs and shaken briefly by hand every 30 minutes. After the acid-leach treatment is completed, the sample is filtered on a Buchner funnel with Whatman 541 paper and washed with about 7.5 L deionized water. Thereafter, the acid-leached sample is dried at 110° C. for 22 hrs.

Third, acid-leach treated AR-glass undergoes an IEX treatment. In this example, ammonium metatungstate, (NH₄)₆H₂W₁₂O₄₀.nH₂O, is used to prepare 3 L 0.05 wt. % tungsten solution for ion exchange (“IEX solution”). About 15.01 g of acid-leach treated AR-glass is added to the IEX solution (“glass/IEX mixture”). The pH of the glass/IEX mixture is measured. As needed, the pH of the mixture is adjusted with a continuous drop-wise addition of about 29.8 wt. % ammonium hydroxide (NH₄OH) to pH 8. The glass/IEX mixture is transferred to a 4-L wide neck plastic container. The plastic container is placed in an air draft oven at 50° C. oven for 2 hrs and shaken briefly by hand every 30 minutes. At the completion of the 2 hr. heating period, the glass/IEX mixture is filtered and IEX-glass sample collected on a Buchner funnel with Whatman 541 paper and washed with about 5 L of deionized water. Thereafter, the IEX-glass sample is dried at 110° C. for 22 hrs.

Fourth, the IEX-glass undergoes a calcination treatment in which the IEX-glass is calcined at 500° C. for 4 hrs in air flow at a rate of 2 L/hr.

The sample is analyzed by ICP-AES, which is expected to result in a tungsten concentration of about 0.01 wt. %.

### Catalyst Composition with A-Glass Substrate

### Example 14

### Platinum on A-06F Glass

A-06F-glass fibers having a mean diameter of 500-600 nm produced by Lauscha Fiber International, is obtained.

First, the as-received, non-calcined A-06F glass sample undergoes an acid-leach treatment. About 21 g of the A-06F glass and 4 L 5.5 wt. % nitric acid are each placed in a 4 L wide neck plastic container. The plastic container is placed in an air draft oven at 90° C. oven for 2 hrs and shaken briefly by hand every 30 minutes. After the acid-leach treatment is completed, the sample is filtered on a Buchner funnel with Whatman 541 paper and washed with about 7.6 L deionized water. Thereafter, the acid-leached sample is dried at 110° C. for 22 hrs.

Second, the acid-leach treated A-06F glass undergoes an IEX treatment. In this example, platinum tetraamine-chloride, [Pt(NH₃)₄](Cl)₂, is used to prepare 1 L 0.01 wt. % platinum solution for ion exchange (“IEX solution”). 20 g of A-06F glass is added to the IEX solution (“glass/IEX mixture”). The pH of the glass/IEX mixture is measured. As needed, the pH of the mixture is adjusted with a continuous drop-wise addition of about 29.8 wt. % ammonium hydroxide (NH₄OH) to greater than pH 10 (in this example, resulting in a pH of about 11.1. The glass/IEX mixture is transferred to a 2-L wide neck plastic container. The container is placed in an air-draft oven at 100° C. oven for 23 hrs. The container is shaken several times over the 23 hr heating period. After the IEX treatment is completed, the glass/IEX mixture is filtered and IEX-glass sample collected on a Buchner funnel with Whatman 541 paper and washed with about 7.6 L of a dilute NH₄OH solution. The dilute NH₄OH solution is prepared by mixing 10 g of a concentrated 29.8 wt. % NH₄OH solution with about 3.8 L of deionized water. Thereafter, the IEX-glass sample is dried at 110° C. for 22 hrs.

Third, the IEX-glass sample undergoes a reducing treatment in which the ion-exchanged sample is reduced at 300° C. for 4 hrs in hydrogen (H₂) under a H₂ flow rate of 2 L/hr.

The sample is analyzed by ICP-AES, resulting in a platinum concentration of about 0.96 wt. %.

### Example 15

### Palladium on A-06F Glass

A-06F-glass fibers having a mean diameter of 500-600 nm produced by Lauscha Fiber International, is obtained.

First, the as-received, non-calcined A-06F glass sample undergoes an acid-leach treatment. About 50 g of the A-06F glass and 4 L 5.5 wt. % nitric acid are each placed in a 4-L wide neck plastic container. The plastic container is placed in an air draft oven at 90° C. oven for 2 hrs and shaken briefly by hand every 30 minutes. After the acid-leach treatment is completed, the sample is filtered on a Buchner funnel with Whatman 541 paper and washed with about 7.6 L deionized water. Thereafter, the acid-leached sample is dried at 110° C. for 22 hrs.

Second, the acid-leach treated A-06F-glass sample undergoes an IEX treatment. In this example, palladium tetraamine-hydroxide, [Pd(NH₃)₄](OH)₂, is used to prepare 3 L 0.001 wt. % palladium solution for ion exchange (“IEX solution”). About 10 g A-06F glass is added to the IEX solution (“glass/IEX mixture”). The pH of the glass/IEX mixture is measured. As needed, the pH of the mixture is adjusted with a continuous drop-wise addition of about 29.8 wt. % ammonium hydroxide (NH₄OH) to greater than pH 10 (in this example, resulting in a pH of about 10.5). The glass/IEX mixture is transferred to a 4-L wide neck plastic container. The container is placed in an air-draft oven at 50° C. oven for 2 hrs and shaken briefly by hand every 30 minutes. After the IEX treatment is completed, the glass/IEX mixture is filtered on a Buchner funnel with Whatman 541 paper and a filtercake is obtained, which is remixed with about 3 L of a dilute NH₄OH solution and filtered again. This remixing/filtering step is repeated two times. The dilute NH₄OH solution is prepared by mixing 10 g of a concentrated 29.8 wt. % NH₄OH solution with about 3.8 L of deionized water. Thereafter, the IEX-glass sample is dried at 110° C. for 22 hrs.

Third, the IEX-glass sample undergoes a reducing treatment in which the IEX-glass sample is reduced at 300° C. for 4 hrs in hydrogen (H₂) under a H₂ flow rate of 2 L/hr.

The sample is analyzed by ICP-AES, resulting in a palladium concentration of about 0.062 wt. %.

### Example 16

### Palladium on A-06F Glass

A-06F-glass fibers having a mean diameter of 500-600 nm produced by Lauscha Fiber International, is obtained.

First, the as-received, non-calcined A-06F glass sample undergoes an acid-leach treatment. About 51 g of the A-06F glass and 4 L 5.5 wt. % nitric acid are each placed in a 4-L wide neck plastic container. The plastic container is placed in an air draft oven at 90° C. oven for 2 hrs and shaken briefly by hand every 30 minutes. After the acid-leach treatment is completed, the sample is filtered on a Buchner funnel with Whatman 541 paper and washed with about 7.6 L deionized water. Thereafter, the acid-leached sample is dried at 110° C. for 22 hrs.

Second, the acid-leach treated A-06F glass undergoes Na⁺-back-ion exchange (“Na—BIX”) treatment. The acid-leached sample from the first step is mixed with 4 L 3 mol/L sodium chloride (NaCl) solution (“glass/NaCl mixture”). The pH of the glass/NaCl mixture is measured. As needed, the pH of the glass/NaCl mixture is adjusted with a continuous drop-wise addition of about 40 wt. % tetrapropylammonium hydroxide to greater than pH 10 (in this example, resulting in a pH of about 10.9). The glass/NaCl mixture is transferred to a 4-L wide-neck plastic container. The plastic container is subsequently placed in an air-draft oven at 50° C. for 4 hrs and shaken briefly by hand every 30 minutes. After the Na—BIX treatment is completed, the glass/NaCl mixture is filtered and the Na—BIX/A-06F sample collected on a Buchner funnel with Whatman 541 paper and washed with about 7.6 L deionized water. Thereafter, the Na—BIX/A-06F-glass sample is dried at 110° C. for 22 hrs.

Third, Na—BIX/A-06F-glass sample undergoes a second ion-exchange (“IEX-2”) treatment. In this example, palladium tetraamine-chloride, [Pd(NH₃)₄](Cl)₂, is used to prepare 1 L 0.01 wt. % palladium solution for ion exchange (“IEX-2 solution”). 35 g of A-06F glass is added to the IEX-2 solution (“glass/IEX-2 mixture”). The pH of the glass/IEX-2 mixture is measured, resulting in a pH of about 8.5. The glass/IEX-2 mixture is transferred to a 2-L wide neck plastic container. The plastic container is placed in an air-draft oven at 50° C. oven for 4 hrs and shaken briefly by hand every 30 minutes. After the ion-exchange treatment is completed, the glass/IEX-2 mixture is filtered on a Buchner funnel with Whatman 541 paper and the IEX-2-glass sample collected is washed with about 7.6 L of a dilute NH₄OH solution. The dilute NH₄OH solution is prepared by mixing 10 g of a concentrated 29.8 wt. % NH₄OH solution with about 3.8 L of deionized water. Thereafter, the ion-x2 sample is dried at 110° C. for 22 hrs.

Fourth, the IEX-2-glass sample undergoes a reducing treatment in which the sample is reduced at 300° C. for 4 hrs in hydrogen (H₂) under a H₂ flow rate of 2 L/hr.

The sample is analyzed by ICP-AES, resulting in a palladium concentration of about 0.09 wt. %.

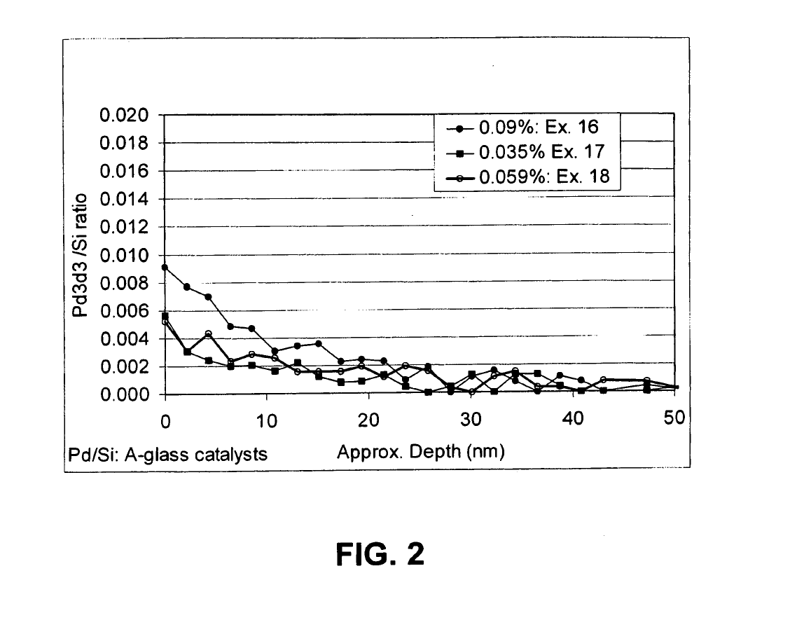

The sample is analyzed by an XPS Sputter Depth Profiling method (as described below), demonstrating, as depicted in FIG. 2, that the thickness of the region in which a substantial portion of the palladium is detected by this method is about 15 nm.

### Example 17

### Palladium on A-06F Glass

A-06F-glass fibers having a mean diameter of 500-600 nm produced by Lauscha Fiber International, is obtained.

First, the A-06F-glass fiber undergoes an IEX treatment. In this example, palladium tetraamine-hydroxide, [Pd(NH₃)₄](OH)₂, is used to prepare 2 L 0.001 wt. % palladium solution for ion exchange (“IEX solution”). About 5.4 g A-06F glass is added to the IEX solution (“glass/IEX mixture”). The pH of the glass/IEX mixture is measured. As needed, the pH of the mixture is adjusted with a continuous drop-wise addition of about 29.8 wt. % ammonium hydroxide (NH₄OH) to greater than pH 10 (in this example, resulting in a pH of about 10.1). The glass/IEX mixture is transferred to a 4-L glass beaker container and placed on a hotplate. The container is mechanically stirred at 59° C. oven for 2 hrs. After the IEX treatment is completed, the glass/IEX mixture is filtered on a Buchner funnel with Whatman 541 paper and a filtercake is obtained, which is remixed with about 3 L of a dilute NH₄OH solution and filtered again. This remixing/filtering step is repeated two times. The dilute NH₄OH solution is prepared by mixing 10 g of a concentrated 29.8 wt. % NH₄OH solution with about 3.8 L of deionized water. Thereafter, the IEX-glass sample is dried at 100° C. for 22 hrs.

Second, the IEX-glass sample undergoes a reducing treatment in which the IEX-glass sample is reduced at 300° C. for 4 hrs in hydrogen (H₂) under a H₂ flow rate of 2 L/hr.

The sample is analyzed by ICP-AES, resulting in a palladium concentration of about 0.035 wt. %.

The sample is analyzed by an XPS Sputter Depth Profiling method (as described below), demonstrating, as depicted in FIG. 2, that the thickness of the region in which a substantial portion of the palladium is detected by this method is about 15 nm.

### Example 18

### Palladium on A-06F Glass

A-06F-glass fibers having a mean diameter of 500-600 nm produced by Lauscha Fiber International, is obtained.

First, the as-received, non-calcined A-06F glass sample undergoes an acid-leach treatment. About 50 g of the A-06F glass and 4 L 5.5 wt. % nitric acid are each placed in a 4-L wide neck plastic container. The plastic container is placed in an air draft oven at 90° C. oven for 2 hrs and shaken briefly by hand every 30 minutes. After the acid-leach treatment is completed, the sample is filtered on a Buchner funnel with Whatman 541 paper and washed with about 7.6 L deionized water. Thereafter, the acid-leached sample is dried at 110° C. for 22 hrs.

Second, the acid-leach treated A-06F-glass sample undergoes an IEX treatment. In this example, palladium tetraamine-hydroxide, [Pd(NH₃)₄](OH)₂, is used to prepare 3 L 0.001 wt. % palladium solution for ion exchange (“IEX-solution”). About 10 g A-06F glass is added to the IEX solution (“glass/IEX mixture”). The pH of the glass/IEX mixture is measured. As needed, the pH of the mixture is adjusted with a continuous drop-wise addition of about 29.8 wt. % ammonium hydroxide (NH₄OH) to greater than pH 10 (in this example, resulting in a pH of about 10.5). The glass/IEX mixture is transferred to a 4-L wide neck plastic container. The container is placed in an air-draft oven at 50° C. oven for 2 hrs and shaken briefly by hand every 30 minutes. After the IEX treatment is completed, the glass/IEX mixture is filtered on a Buchner funnel with Whatman 541 paper and a filtercake is obtained, which is remixed with about 3 L of a dilute NH₄OH solution and filtered again. This remixing/filtering step is repeated two times. The dilute NH₄OH solution is prepared by mixing 10 g of a concentrated 29.8 wt. % NH₄OH solution with about 3.8 L of deionized water. Thereafter, the IEX-glass sample is dried at 110° C. for 22 hrs.

Third, the IEX-glass sample undergoes a reducing treatment in which the IEX-glass is initially calcined at 300° C. for 2 hrs in air at an air flow rate of 2 L/hr and thereafter reduced at 300° C. for 4 hrs in hydrogen (H₂) under a H₂ flow rate of 2 L/hr.

The sample is analyzed by ICP-AES, resulting in a palladium concentration of about 0.059 wt. %.

The sample is analyzed by an XPS Sputter Depth Profiling method (as described below), demonstrating, as depicted in FIG. 2, that the thickness of the region in which a substantial portion of the palladium is detected by this method is about 15 nm.

### Example 19

### Palladium on A-06F Glass

A-06F-glass fibers having a mean diameter of 500-600 nm produced by Lauscha Fiber International, is obtained.

First, the as-received, non-calcined A-06F glass sample undergoes an acid-leach treatment. About 8.43 g of the A-06F glass and 1.5 L 5.5 wt. % nitric acid are each placed in a 2-L glass beaker and mechanically stirred with a stainless steel paddle stirrer at 300-500 rpm at 22° C. for 30 min. After the acid-leach treatment is completed, the sample is filtered on a Buchner funnel with Whatman 541 paper and washed with about 7.6 L deionized water. Thereafter, the acid-leached sample is dried at 110° C. for 22 hrs.

Second, the acid-leach treated A-06F-glass sample undergoes an IEX treatment. In this example, palladium tetraamine-hydroxide, [Pd(NH₃)₄](OH)₂, is used to prepare 500 mL 0.01 wt. % palladium solution for ion exchange (“IEX solution”). About 4.2 g A-06F glass is added to the IEX solution (“glass/IEX mixture”). The pH of the glass/IEX mixture is measured. As needed, the pH of the mixture is adjusted with a continuous drop-wise addition of about 29.8 wt. % ammonium hydroxide (NH₄OH) to greater than pH 10 (in this example, resulting in a pH of about 10.2). The glass/IEX mixture is transferred to a 1-L beaker and stirred at 50° C. for 2 hrs. After the IEX treatment is completed, the glass/IEX mixture is filtered on a Buchner funnel with Whatman 541 paper and washed with about 7.6 L deionized water. Thereafter, the IEX-glass sample is dried at 110° C. for 22 hrs.

Third, the IEX-glass sample undergoes a reducing treatment in which the IEX-glass is initially calcined at 300° C. for 2 hrs in air at an air flow rate of 2 L/hr and thereafter reduced at 300° C. for 4 hrs in hydrogen (H₂) under a H₂ flow rate of 2 L/hr.

The sample is analyzed by ICP-AES, resulting in a palladium concentration of about 0.57 wt. %.

### Example 20

### Platinum on A-06F Glass

A-06F-glass fibers having a mean diameter of 500-600 nm produced by Lauscha Fiber International, is obtained.

First, the as-received, non-calcined A-06F glass sample undergoes an acid-leach treatment. About 30 g of the A-06F glass and 4 L 5.5 wt. % nitric acid are each placed in a 4-L wide neck plastic container. The plastic container is placed in an air draft oven at 90° C. oven for 2 hrs and shaken briefly by hand every 30 minutes. After the acid-leach treatment is completed, the sample is filtered on a Buchner funnel with Whatman 541 paper and washed with about 7.6 L deionized water. Thereafter, the acid-leached sample is dried at 110° C. for 22 hrs.

Second, the acid-leach treated A-06F glass undergoes an IEX treatment. In this example, platinum tetraamine-chloride, [Pt(NH₃)₄](Cl)₂, is used to prepare 3 L 0.01 wt. % platinum solution for ion exchange (“IEX solution”). 15.1 g of acid-leached A-06F glass is added to the IEX solution (“glass/IEX mixture”). The pH of the glass/IEX mixture is measured. As needed, the pH of the mixture is adjusted with a continuous drop-wise addition of about 29.8 wt. % ammonium hydroxide (NH₄OH) to greater than pH 10 (in this example, resulting in a pH of about 10.07). The glass/IEX mixture is transferred to a 4-L wide neck plastic container. The container is placed in an air-draft oven at 50° C. oven for 2 hrs. The container is shaken briefly by hand every 30 minutes. After the IEX treatment is completed, the glass/IEX mixture is filtered and IEX-glass sample collected on a Buchner funnel with Whatman 541 paper and washed with about 7.6 L of a dilute NH₄OH solution. The dilute NH₄OH solution is prepared by mixing 10 g of a concentrated 29.8 wt. % NH₄OH solution with about 3.8 L of deionized water. Thereafter, the IEX-glass sample is dried at 110° C. for 22 hrs.

Third, the IEX glass sample undergoes a reducing treatment in which the sample is reduced at 300° C. for 4 hrs in hydrogen (H₂) under a H₂ flow rate of 2 L/hr.

The sample is analyzed by ICP-AES, resulting in a platinum concentration of about 0.33 wt. %.

### Example 21

### Platinum on A-06F Glass

A-06F-glass fibers having a mean diameter of 500-600 nm produced by Lauscha Fiber International, is obtained.

First, the as-received, non-calcined A-06F glass sample undergoes an acid-leach treatment. About 30 g of the A-06F glass and 4 L 5.5 wt. % nitric acid are each placed in a 4-L wide neck plastic container. The plastic container is placed in an air draft oven at 90° C. oven for 2 hrs and shaken briefly by hand every 30 minutes. After the acid-leach treatment is completed, the sample is filtered on a Buchner funnel with Whatman 541 paper and washed with about 7.6 L deionized water. Thereafter, the acid-leached sample is dried at 110° C. for 22 hrs.

Second, the acid-leach treated A-06F glass undergoes an IEX treatment. In this example, platinum tetraamine-chloride, [Pt(NH₃)₄](Cl)₂, is used to prepare 3 L 0.01 wt. % platinum solution for ion exchange (“IEX solution”). 9.3 g of acid-leached A-06F glass is added to the IEX solution (“glass/IEX mixture”). The pH of the glass/IEX mixture is measured. As needed, the pH of the mixture is adjusted with a continuous drop-wise addition of about 40 wt. % tetrapropylammonium hydroxide to greater than pH 10 (in this example, resulting in a pH of about 11.07). The glass/IEX mixture is transferred to a 4-L wide neck plastic container. The container is placed in an air-draft oven at 100° C. oven for 22 hrs. The container is shaken briefly by hand every 30 minutes. After the IEX treatment is completed, the glass/IEX mixture is filtered and IEX-glass sample collected on a Buchner funnel with Whatman 541 paper and washed with about 7.6 L of a dilute NH₄OH solution. The dilute NH₄OH solution is prepared by mixing 10 g of a concentrated 29.8 wt. % NH₄OH solution with about 3.8 L of deionized water. Thereafter, the IEX-glass sample is dried at 110° C. for 22 hrs.

Third, the IEX glass sample undergoes a reducing treatment in which the sample is reduced at 300° C. for 4 hrs in hydrogen (H₂) under a H₂ flow rate of 2 L/hr.

The sample is analyzed by ICP-AES, resulting in a platinum concentration of about 0.59 wt. %.

### Example 22

### Platinum on A-06F Glass

A-06F-glass fibers having a mean diameter of 500-600 nm produced by Lauscha Fiber International, is obtained.

First, the as-received, non-calcined A-06F glass sample undergoes an acid-leach treatment. About 30 g of the A-06F glass and 4 L 5.5 wt. % nitric acid are each placed in a 4-L wide neck plastic container. The plastic container is placed in an air draft oven at 90° C. oven for 2 hrs and shaken briefly by hand every 30 minutes. After the acid-leach treatment is completed, the sample is filtered on a Buchner funnel with Whatman 541 paper and washed with about 7.6 L deionized water. Thereafter, the acid-leached sample is dried at 110° C. for 22 hrs.

Second, the acid-leach treated A-06F glass undergoes an IEX treatment. In this example, platinum tetraamine-chloride, [Pt(NH₃)₄](Cl)₂, is used to prepare 3 L 0.01 wt. % platinum solution for ion exchange (“IEX solution”). 21 g of acid-leached A-06F glass is added to the IEX solution (“glass/IEX mixture”). The pH of the glass/IEX mixture is measured. As needed, the pH of the mixture is adjusted with a continuous drop-wise addition of about 29.8 wt. % ammonium hydroxide (NH₄OH) to greater than pH 10 (in this example, resulting in a pH of about 10.38). The glass/IEX mixture is transferred to a 4-L wide neck plastic container. The container is placed in an air-draft oven at 100° C. oven for 22 hrs. The container is shaken briefly by hand every 30 minutes. After the IEX treatment is completed, the glass/IEX mixture is filtered and IEX-glass sample collected on a Buchner funnel with Whatman 541 paper and washed with about 7.6 L of a dilute NH₄OH solution. The dilute NH₄OH solution is prepared by mixing 10 g of a concentrated 29.8 wt. % NH₄OH solution with about 3.8 L of deionized water. Thereafter, the IEX-glass sample is dried at 110° C. for 22 hrs.

Third, the IEX glass sample undergoes a reducing treatment in which the sample is reduced at 300° C. for 4 hrs in hydrogen (H₂) under a H₂ flow rate of 2 L/hr.

The sample is analyzed by ICP-AES, resulting in a platinum concentration of about 0.71 wt. %.

### Example 23

### Palladium & Copper on A-06F Glass

A-06F-glass fibers having a mean diameter of 500-600 nm produced by Lauscha Fiber International, is obtained.

First, the as-received, non-calcined A-06F glass sample undergoes an acid-leach treatment. 15 g of the A-06F glass and 4 L 5.5 wt. % nitric acid are each placed in a 4-L wide neck plastic container. The plastic container is placed in an air draft oven at 90° C. oven for 2 hrs and shaken briefly by hand every 30 minutes. After the acid-leach treatment is completed, the sample is filtered on a Buchner funnel with Whatman 541 paper and washed with about 7.6 L deionized water. Thereafter, the acid-leached sample is dried at 110° C. for 22 hrs.

Second, the acid-leach treated A-06F glass undergoes a double-IEX treatment. In this example, 3 L 0.0005 wt. % total metal solution is used for double-IEX (“double-IEX solution”). The double IEX solution is prepared by mixing 1.5 L 0.0005 wt. % palladium solution and 1.5 L 0.0005 wt. % copper solution. In this example, palladium tetraamine hydroxide is used to prepare 1.5 L 0.0005 wt. % palladium solution and copper nitrate is used to prepare 1.5 L 0.0005 wt. % copper solution About 14 g of A-06F glass is added to the double-IEX solution (“glass/IEX mixture”). The pH of the glass/IEX mixture is measured. As needed, the pH of the mixture is adjusted with a continuous dropwise addition of about 29.8 wt. % ammonium hydroxide (NH₄OH) to greater than pH 10 (in this example, resulting in a pH of about 10.9). The glass/IEX mixture is transferred to a 4-L wide neck plastic container. The container is placed in an air-draft oven at 50° C. oven for 2 hrs and shaken briefly by hand every 30 minutes. After the double-IEX treatment is completed, the glass/IEX mixture is filtered on a Buchner funnel with Whatman 541 paper and double-IEX-glass sample collected is washed with about 7.6 L of a dilute NH₄OH solution. The dilute NH₄OH solution is prepared by mixing 10 g of a concentrated 29.8 wt. % NH₄OH solution with about 3.8 L of deionized water. Thereafter, the double-IEX-glass sample is dried at 110° C., for 22 hrs.

Third, the double-IEX-glass sample undergoes a reducing treatment in which the double-IEX-glass sample is reduced at 300° C. for 4 hrs in hydrogen (H₂) under a H₂ flow rate of 2 L/hr.

The sample is analyzed by ICP-AES, resulting in a palladium concentration of about 0.019 wt. % and a copper concentration of about 0.02 wt. %.

### Example 24

### Silver on A-06F Glass

A-06F-glass fibers having a mean diameter of 500-600 nm produced by Lauscha Fiber International, is obtained.

First, the as-received, non-calcined A-06F glass sample undergoes an acid-leach treatment. About 51 g of the A-06F glass and 4 L 5.5 wt. % nitric acid are each placed in a 4-L wide neck plastic container. The plastic container is placed in an air draft oven at 90° C. oven for 2 hrs and shaken briefly by hand every 30 minutes. After the acid-leach treatment is completed, the sample is filtered on a Buchner funnel with Whatman 541 paper and washed with about 7.6 L deionized water. Thereafter, the acid-leached sample is dried at 110° C. for 22 hrs.

Second, the acid-leach treated A-06F glass undergoes an IEX treatment. In this example, silver nitrate is used to prepare 4 L 0.001 wt. % silver solution for ion exchange (“IEX solution”). 10 g of A-06F glass is added to the IEX solution (“glass/IEX mixture”). The pH of the glass/IEX mixture is measured. As needed, the pH of the mixture is adjusted with a continuous drop-wise addition of about 29.8 wt. % ammonium hydroxide (NH₄OH) to greater than pH 11 (in this example, resulting in a pH of about 11.5). The glass/IEX mixture is transferred to a 4-L wide neck plastic container. The plastic container is placed in an air-draft oven at 50° C. oven for 2 hrs and shaken briefly by hand every 30 minutes. After the IEX treatment is completed, glass/IEX mixture is filtered and the IEX-glass sample collected on a Buchner funnel with Whatman 541 paper and washed with about 7.6 L of a dilute NH₄OH solution. The dilute NH₄OH solution is prepared by mixing 10 g of a concentrated 29.8 wt. % NH₄OH solution with about 3.8 L of deionized water. Thereafter, the IEX-glass sample is dried at 110° C. for 22 hrs.

Third, the IEX-glass sample undergoes a reducing treatment in which the IEX-glass sample is reduced at 300° C. for 4 hrs in hydrogen (H₂) under a H₂ flow rate of 2 L/hr.

The sample is analyzed by ICP-AES, resulting in a silver concentration of about 0.053 wt. %.

### Example 25

### Platinum on A-06F Glass

A-06F-glass fibers having a mean diameter of 500-600 nm produced by Lauscha Fiber International, is obtained.

First, the as-received, non-calcined A-06F glass sample undergoes an acid-leach treatment. About 100 g of the A-06F glass and 4 L 5.5 wt. % nitric acid are each placed in a 4-L wide neck plastic container. The plastic container is placed in an air draft oven at 90° C. oven for 2 hrs and shaken briefly by hand every 30 minutes. After the acid-leach treatment is completed, the sample is filtered on a Buchner funnel with Whatman 541 paper and washed with about 7.6 L deionized water. Thereafter, the acid-leached sample is dried at 110° C. for 22 hrs.

Second, the acid-leach treated A-06F glass undergoes an IEX treatment. In this example, platinum tetraamine-chloride, [Pt(NH₃)₄](Cl)₂, is used to prepare 3 L 0.016 wt. % platinum solution for ion exchange (“IEX solution”). 48.17 g of A-06F glass is added to the IEX solution (“glass/IEX mixture”). The pH of the glass/IEX mixture is measured. As needed, the pH of the mixture is adjusted with a continuous drop-wise addition of about 29.8 wt. % ammonium hydroxide (NH₄OH) to greater than pH 10 (in this example, resulting in a pH of about 10.06). The glass/IEX mixture is transferred to a 4-L wide neck plastic container. The container is placed in an air-draft oven at 50° C. oven for 2 hrs. The container is shaken briefly by hand every 30 minutes. After the IEX treatment is completed, the glass/IEX mixture is filtered and IEX-glass sample collected on a Buchner funnel with Whatman 541 paper and washed with about 7.6 L of a dilute NH₄OH solution. The dilute NH₄OH solution is prepared by mixing 10 g of a concentrated 29.8 wt. % NH₄OH solution with about 3.8 L of deionized water. Thereafter, the IEX-glass sample is dried at 110° C. for 22 hrs.

Third, the IEX glass sample undergoes a reducing treatment in which the sample is reduced at 500° C. for 4 hrs in hydrogen (H₂) under a H₂ flow rate of 2 L/hr.

The sample is analyzed by ICP-AES, resulting in a platinum concentration of about 0.147 wt. %.

### Example 26

Platinum on A-06F Glass

A-06F-glass fibers having a mean diameter of 500-600 nm produced by Lauscha Fiber International, is obtained.

First, the as-received, non-calcined A-06F glass sample undergoes an acid-leach treatment. About 21 g of the A-06F glass and 4 L 5.5 wt. % nitric acid are each placed in a 4-L wide neck plastic container. The plastic container is placed in an air draft oven at 90° C. oven for 2 hrs and shaken briefly by hand every 30 minutes. After the acid-leach treatment is completed, the sample is filtered on a Buchner funnel with Whatman 541 paper and washed with about 7.6 L deionized water. Thereafter, the acid-leached sample is dried at 110° C. for 22 hrs.

Second, the acid-leach treated A-06F glass undergoes an IEX treatment. In this example, platinum tetraamine-chloride, [Pt(NH₃)₄](Cl)₂, is used to prepare 4 L 0.02 wt. % platinum solution for ion exchange (“IEX solution”). About 21 g of acid-leached A-06F glass is added to the IEX solution (“glass/IEX mixture”). The pH of the glass/IEX mixture is measured. As needed, the pH of the mixture is adjusted with a continuous drop-wise addition of about 29.8 wt. % ammonium hydroxide (NH₄OH) to greater than pH 10 (in this example, resulting in a pH of about 10.90). The glass/IEX mixture is transferred to a 4-L wide neck plastic container. The container is placed in an air-draft oven at 100° C. oven for 22 hrs. The container is shaken briefly by hand every 30 minutes. After the IEX treatment is completed, the glass/IEX mixture is filtered and IEX-glass sample collected on a Buchner funnel with Whatman 541 paper and washed with about 7.6 L of a dilute NH₄OH solution. The dilute NH₄OH solution is prepared by mixing 10 g of a concentrated 29.8 wt. % NH₄OH solution with about 3.8 L of deionized water. Thereafter, the IEX-glass sample is dried at 110° C. for 22 hrs.

Third, the IEX glass sample undergoes a reducing treatment in which the sample is reduced at 300° C. for 4 hrs in hydrogen (H₂) under a H₂ flow rate of 2 L/hr.

The sample is analyzed by ICP-AES, resulting in a platinum concentration of about 0.67 wt. %.

### Example 27

### Palladium on E-06F Glass Non-Leached

E-06F-glass fibers having a mean diameter of 500-600 nm produced by Lauscha Fiber International, is obtained.

First, the unleached E-06F-glass sample undergoes an IEX treatment. In this example, palladium tetraamine-hydroxide, [Pd(NH₃)₄](OH)₂, is used to prepare 2 L 0.00008 wt. % palladium solution for ion exchange (“IEX solution”). About 15.45 g E-06F glass is added to the IEX solution (“glass/IEX mixture”). The pH of the glass/IEX mixture is measured. As needed, the pH of the mixture is adjusted with a continuous drop-wise addition of about 29.8 wt. % ammonium hydroxide (NH₄OH) to greater than pH 10 (in this example, resulting in a pH of about 10.99). The glass/IEX mixture is transferred to a 4-L wide neck plastic container. The container is placed in an air-draft oven at 50° C. oven for 2 hrs. The container is shaken briefly by hand every 30 minutes. After the IEX treatment is completed, the glass/IEX mixture is filtered and IEX-glass sample collected on a Buchner funnel with Whatman 541 paper and washed with about 7.6 L of a dilute NH₄OH solution. The dilute NH₄OH solution is prepared by mixing 10 g of a concentrated 29.8 wt. % NH₄OH solution with about 3.8 L of deionized water. Thereafter, the IEX-glass sample is dried at 110° C. for 22 hrs.

Second, the IEX-glass undergoes a reducing treatment in which the IEX-glass is reduced at 300° C. for 4 hrs in hydrogen (H₂) under a H₂ flow rate of 2 L/hr.

The sample is analyzed by ICP-AES, resulting in a palladium concentration of about 0.014 wt. %.

### Example CH-1

### Analytical Methods Re/XPS Sputtering, SARC_{Na}, Isoelectric Point (IEP) and S.A._{N2-BET} or S.A._{Kr-BET} Determination

### X-Ray Photoelectron Spectroscopy (XPS) Sputter Depth Profiling Method

The XPS Sputter Depth Profiles are obtained using a PHI Quantum 200 Scanning ESCA Microprobe™ (Physical Electronics, Inc.) with a micro-focused, monochromatized Al Kα X-ray source at 1486.7 eV. A dual neutralization capability using low energy electrons and positive ions to provide charge compensation during spectral acquisition is standard in this instrument.

XPS spectra are generally measured under the following conditions:
X-ray beam diameter 10-200 μm
X-ray beam power 2-40 W
Sample analysis area 10-200 μm
Electron emission angle 45° to sample normal

All XPS spectra and sputter depth profiles are recorded at room temperature without sample pretreatment, with the exception of introducing the samples in the vacuum environment of the XPS instrument.

Sputter depth profiles are generated by alternating cycles of spectral acquisition of the sample surface, followed by 2 kV Ar⁺ sputtering of the sample surface for 15-30 s in each cycle to remove surface material. The sputter depth rate is calibrated using a silica thin film of known thickness.

Atomic concentration values for Pd and Si shown in FIGS. 1 and 2 are obtained by taking the Pd 3d_{3/2} and Si 2p peak areas and correcting for their respective atomic sensitivity factors and the analyzer transmission function.

As will be understood by those skilled in the art of XPS analysis, the determination of the sputter depth parameter is subject to both human and mechanical error, which in combination can impose an uncertainty of about 25% on each reported value of sputter depth determined by the XPS Sputter Depth Profile technique. Accordingly, this uncertainty is manifested in the values of the depth indicated in FIGS. 1 and 2. This imprecision is general throughout the art of XPS analysis and is not sufficient to preclude the differentiation between the catalyst compositions described herein or from other compositions not otherwise described and claimed here, in view of the mean thickness of the catalytically active region, among other material attributes disclosed herein.

### Transmission Electron Microscopy (TEM) Analytical Method

Transmission electron microscopy (TEM) examination of samples is performed using a JEOL 3000F Field Emission scanning transmission electron microscopy (STEM) instrument operated at 300 kV accelerating voltage. The instrument is equipped with an Oxford Instruments Inca X-ray spectroscopy system for conducting local chemical analysis using energy dispersive spectroscopy.

Samples are prepared by first embedding the sample material in a standard embedding epoxy known to those skilled in the art of TEM analysis. After curing, the epoxy-embedded sample material is sectioned using an ultra-microtome sectioning device to produce ˜80 nm thick sections. Sections are collected on thin film holey carbon supports and, without further processing, are properly oriented in the electron-beam field of the above-described STEM instrument for examination and analysis.

As will be understood by those skilled in the art of TEM analysis, the determination of a target analyte's location and the mean thickness of a region of interest versus a substrate's surface using TEM analysis is subject to both human and mechanical error, which can impose uncertainty in the TEM vertical depth measurement (vs. a specific reference point) of about ±20% and a lateral position measurement (vs. a specific reference point) of about ±5%, depending the sample's image resolution, target analyte's physicochemical characteristics and sample morphology, among other factors. Accordingly, the uncertainty is manifested in the distance measured for the catalytic constituent vs. the sample substrate surface. This imprecision is general throughout the art of TEM analysis and is not sufficient to preclude differentiation between catalyst compositions.

### SARC_{Na} Determination, Blank for SARC_{Na} and Related Statistical Analysis

The sodium surface area rate of change (“SARC_{Na}”) is reported as a ratio of NaOH titrant volumes for reasons discussed above.

A SARC_{Na} is determined for each of the samples specified below in the following examples according to the procedure described above for SARC_{Na}. A blank sample is prepared by producing a 3.5M NaCl solution (i.e., 30 g NaCl in 150 mL deionized water), but contains no substrate sample. However, to account for statistical variability in the SARC_{Na} experimental procedure, four independent blank samples are titrated and the mean value of the titrant volumes for the specified concentration (0.01N in this case) used to obtain a Vᵢ and V_{5 to 15} (i.e., Vₜₒₜₐₗ-Vᵢ) are used to adjust (i.e., correct) the volume of titrant used in the SARC_{Na} determination of each substrate sample. The blank sample is pH adjusted and titrated according to the same procedure described above for SARC_{Na} determinations, but again, without substrate present.

A statistical analysis of the blank titrant volumes are reported in the table of analytical test results, provided below, for each blank sample run and its respective mean and standard deviation, or σ, for Vₜₒₜₐₗ. As well, the inherent statistical variations corresponding to each titrant volume, Vᵢ, V₅, V₁₀ and V₁₅, arising from their respective Vₜₒₜₐₗ are also reported accordingly. From a statistical perspective, using the statistical t-distribution, there is a 95% degree of certainty that values outside the indicated confidence interval, around the mean values are reliable and do not arise from deviations inherent to the experimental method itself. So, values of Vᵢ and Vₜ measured for the substrate samples that are within the confidence interval around the blank mean value are considered to be statistically indistinguishable from the blank. Accordingly, SARC_{Na} values are not calculated for such samples.

### Isoelectric Point (IEP) Determination

The isoelectric point (“IEP”) for each of the samples specified below is determined according to the following procedure. IEP measurements are made with a Mettler Toledo SevenMulti meter with pH mv/ORP module, fitted with a Mettler Toledo INLAB 413 pH combination electrode. The instrument is calibrated with standard pH buffer solutions of pH 2, 4, 7 and 10 over the entire IEP range of interest. The IEP is determined for each sample by wetting the samples with an amount of 16 MΩ deionized water (at about 25° C.) sufficient to bring the sample to a state of incipient wetness, which will result in producing a relatively dense aqueous slurry-like or paste-like mixture. In turn, this state of incipient wetness will allow liquid contact of both the glass electrode and its reference electrode junctions with the liquid (in this case, water of the slurry- or paste-like mixture) in contact with the solid sample being tested. This procedure will require variable amounts of water, depending on the form of the sample (e.g. glass micro fiber, granular powder, chopped fibers, etc.) and the extent of its porosity (if any). But in each case, the volume of added water should be only enough to allow sufficient liquid contact with both glass electrode and reference electrode junctions. In other words, adding water beyond a sample's state of incipient wetness should be avoided, to the extent reasonably possible to do so, for the sample being tested. The solid sample is mixed, by hand, with the deionized water (added to produce incipient wetness) using the electrode tip in each case until the measured pH stabilizes, then the resulting pH is read from the meter.

### N₂ BET or Kr BET Surface Area (S.A.) Determination

S.A._{N2-BET} or S.A._{Kr-BET} determinations are made, as appropriate, for each of the samples specified below according to the ASTM procedures referenced above. As discussed more fully above, for higher surface area measurements (e.g., about 3 to 6 m²/g) N₂ BET, according to the method described by ASTM D3663-03, is likely to be the preferred surface area measurement technique. While for lower surface area measurements (e.g., < about 3 m²/g) Kr BET, according to the method described by ASTM D4780-95, (“S.A._{Kr-BET}”), is likely to be the preferred surface area measurement technique.

SARC_{Na} Blank Measurements & Statistical Analysis for Correction of SARC_{Na} Titration Values

| | | | Volume of Titrant (ml) Used in NaOH Titration | | | | | |
|---|---|---|---|---|---|---|---|---|
| | | | to Obtain pH 9.0, from Initial pH 4.0, at tₒ (Vᵢ) and | | | | | |
| | Dilute NaOH | | to Maintain pH 9.0 at t₅, t₁₀ and t₁₅ (V₅ _{to 15}) | | | | | |
| | Titrant | S.A._{N2-BET} | Vᵢ at | V₅ at | V₁₀ at | V₁₅ at | Sum of | Vₜₒₜₐₗ = |
| Sample ID | Conc. (N) | (m²/g) | 0 min. | 5 min. | 10 min. | 15 min. | V_{5 to 15} | Vᵢ + V_{5 to 15} |
| Blank A | 0.01 | N/A | 1.5 | 0.3 | 0.1 | 0.2 | 0.6 | 2.1 |
| Blank B | 0.01 | N/A | 2.2 | 0.1 | 0.1 | 0.2 | 0.4 | 2.6 |
| Blank C | 0.01 | N/A | 2.4 | 0.1 | 0.1 | 0.1 | 0.3 | 2.7 |
| Blank D | 0.01 | N/A | 2.2 | 0.1 | 0.2 | 0.1 | 0.4 | 2.6 |
| Blank Mean | 0.01 | N/A | 2.075 | 0.15 | 0.125 | 0.15 | 0.325 | 2.5 |
| Blank Std. Dev. | 0.01 | N/A | 0.3947 | 0.1 | 0.05 | 0.0577 | N/A | 0.2708 |
| Blank 95% | | | 1.45-2.70 | | | | | 2.07-2.93 |
| Confidence Interval | | | | | | | | |

### Example CH-2

### E-Glass-SARC_{Na}

E-06F glass sample, as glass fibers having a mean diameter of 500-600 nm produced by Lauscha Fiber International, is obtained.

Sample A-1 is the as-received E-glass sample, while A-2 is prepared by calcining, but not leaching, the as-received E-glass. For Samples A-1 and A-2, the non-leached E-glass sample undergoes a calcination heat treatment. In that treatment, the non-leached E-glass is calcined at 600° C. for 4 hrs in air under an air flow rate of 1 L/hr.

Comparative Sample Comp-B is prepared by acid-leach treating the as-received, non-calcined, E-glass. For Comparative Sample Comp-B, about 15 g of the E-glass and 1.5 L 9 wt. % nitric acid are each placed in a 4-L wide-neck plastic container. The plastic container is placed in an air draft oven at 95° C. for 4 hr and shaken briefly by hand every 30 minutes. After the acid-leach treatment is completed, the sample is filtered on a Buchner funnel with Whatman 541 paper and washed with about 7.6 L deionized water. Thereafter, the acid-leached sample is dried at 110° C. for 22 hrs.

Samples A-1, A-2 and Comp-B are analyzed by the Analytical Method for Determining SARC_{Na} described above. The results are presented in the table below.

| | | | Actual Volume of Titrant (ml) Used in NaOH Titration | | | | | |
|---|---|---|---|---|---|---|---|---|
| | | | to Obtain pH 9.0, from Initial pH 4.0, at tₒ (Vᵢ) and | | | | | |
| | | Dilute NaOH | to Maintain pH 9.0 at t₅, t₁₀ and t₁₅ (V_{5 to 15}) | | | | | |
| | | Titrant | Vᵢ at | V₅ at | V₁₀ at | V₁₅ at | | |
| Sample ID | Sample Desc. | Conc. (N) | 0 min. | 5 min. | 10 min. | 15 min. | Vₜₒₜₐₗ | Vₜₒₜₐₗ − Vᵢ |
| Blank | Blank Mean | 0.01 | 2.1 | 0.15 | 0.125 | 0.15 | 2.5 | N/A |
| A-1 | As-recv'd E-06F | 0.01 | 20.5 | 0.5 | 0.4 | 0.3 | 21.7 | 1.2 |
| A-2 | Calcined E-06F | 0.1 | 0.7 | 0 | 0.1 | 0 | 0.8 | 0.1 |
| Comp-B | Leached E-06F | 0.1 | 22.6 | 1.9 | 0.9 | 0.4 | 25.8 | 3.2 |

| | | | | Volume of Titrant (ml) Used in SARC_{Na} | | | | | |
|---|---|---|---|---|---|---|---|---|---|
| | | | | Determination* | | | | | |
| | | | S.A._{N2-BET} | Vᵢ at | V₅ at | V₁₀ at | V₁₅ at | | SARC_{Na} |
| Sample ID | Sample Desc. | IEP | (m²/g) | 0 min. | 5 min. | 10 min. | 15 min. | Vₜₒₜₐₗ | (Vₜₒₜₐₗ − Vᵢ)/Vᵢ |
| Blank | Blank Mean | N/A | N/A | 2.1 | 0.15 | 0.125 | 0.15 | 2.5 | N/A |
| A-1 corrected | As-recv'd E-06F | 8.9 | 2.7 | 18.4 | 0.35 | 0.25 | 0.15 | 19.2 | 0.04 |
| A-2* not corrected | Calcined E-06F | 9.5 | ≦7 | 0.7 | 0 | 0.1 | 0 | 0.8 | <~0.2* |
| Comp-B* not corrected | Leached E-06F | 4.1 | 161 | 22.6 | 1.9 | 0.9 | 0.4 | 25.8 | <~0.2* |

*Blank sample titrations are not used for correcting this sample titration since blank correction values are obtained with NaOH titrant concentration of 0.01N, not 0.1N NaOH titrant used for SARC_{Na} analysis of these particular samples.

### Example CH-3

### AR-Glass-SARC_{Na}

AR-glass Cem-FIL Anti-Crak™ HD, sample, as glass fibers having a mean diameter of about 17-20 microns, produced by Saint-Gobain Vetrotex, is obtained. This glass is used for Samples A, B and C in this example.

ARG 6S-750 glass sample, as glass fibers having a mean diameter of about 13 microns produced by Nippon Electric Glass is obtained. This glass is used for Samples D and E in this example.

Samples A and D are prepared by calcining the as-received AR- and ARG-glass, respectively. For Samples A and D, the AR- and ARG-glass samples undergo a calcination heat treatment. In that treatment, the AR-glass and AR-glass is calcined at 600° C. for 4 hrs in air under an air flow rate of 1 L/hr.

Samples B, C and E are prepared by acid-leach treating the as-received, non-calcined, AR-glass and ARG-glass, respectively.

For Samples B and C, about 101 g of the AR-glass and 4 L 5.5 wt. % nitric acid are each placed in a 4-L wide-neck plastic container. The plastic container is placed in an air draft oven at 90° C. for 2 hr and shaken briefly by hand every 30 minutes. After the acid-leach treatment is completed, the sample is filtered on a Buchner funnel with Whatman 541 paper and washed with about 7.6 L deionized water. Thereafter, the acid-leached sample is dried at 110° C. for 22 hrs.

Similarly, for Sample E, about 58 g of the ARG-glass and 4 L 5.5 wt. % nitric acid are each placed in a 4-L wide-neck plastic container. The plastic container is placed in an air draft oven at 90° C. for 2 hr and shaken briefly by hand every 15 minutes. After the acid-leach treatment is completed, the sample is filtered on a Buchner funnel with Whatman 541 paper and washed with about 7.6 L deionized water. Thereafter, the acid-leached sample is dried at 110° C. for 22 hrs.

Samples A-E are analyzed by the Analytical Method for Determining SARC_{Na} described above. The results are presented in the table below.

| | | | Actual Volume of Titrant (ml) Used in Titration | | | | | |
|---|---|---|---|---|---|---|---|---|
| | | | to Obtain pH 9.0, from pH 4.0, at tₒ (Vᵢ) and | | | | | |
| | | Dilute NaOH | to Maintain pH 9.0 at t₅,t₁₀ and t₁₅ (V_{5 to 15}) | | | | | |
| | | Titrant | Vᵢ at | V₅ at | V₁₀ at | V₁₅ at | | |
| Sample ID | Sample Desc. | Conc. (N) | 0 min. | 5 min. | 10 min. | 15 min. | Vₜₒₜₐₗ | Vₜₒₜₐₗ − Vᵢ |
| Blank | Blank Mean | 0.01 | 2.1 | 0.15 | 0.125 | 0.15 | 2.5 | N/A |
| Blank 95% | Statistical | | 1.44-2.70 | | | | 2.07-2.93 | |
| Confidence Interval | Confidence Interval | | | | | | | |
| A | Calcined AR | 0.01 | 2.4 | 0 | 0 | 0.1 | 2.5 | 0.1 |
| B | Leached AR | 0.01 | 2.2 | 0.1 | 0.1 | 0.1 | 2.5 | 0.3 |
| C | Leached AR | 0.01 | 1.7 | 0.1 | 0.1 | 0.1 | 2.0 | 0.3 |
| D | Calcined ARG 6S-750 | 0.01 | 1.6 | 0.4 | 0.3 | 0.4 | 2.7 | 1.1 |
| E | Leached ARG 6S-750 | 0.01 | 2.1 | 0.2 | 0.1 | 0.1 | 2.5 | 0.4 |

| | | | | Corrected Volume of Titrant (ml) Used in | | | | | |
|---|---|---|---|---|---|---|---|---|---|
| | | | | SARC_{Na} Determination | | | | | Corrected |
| | | | S.A._{Kr-BET} | Vᵢ at | V₅ at | V₁₀ at | V₁₅ at | | SARC_{Na} |
| Sample ID | Sample Desc. | IEP | (m²/g) | 0 min. | 5 min. | 10 min. | 15 min. | Vₜₒₜₐₗ | (Vₜₒₜₐₗ − Vᵢ)/Vᵢ |
| Blank | Blank Mean | N/A | N/A | 2.1 | 0.15 | 0.125 | 0.15 | 2.5 | N/A |
| A corrected | Calcined AR | 9.9 | 0.13 | 0.30 | −0.15 | −0.13 | −0.05 | 0 | N/A^{†} |
| B corrected | Leached AR | 9.6 | 0.16 | 0.10 | −0.05 | −0.03 | −0.05 | 0 | N/A^{†} |
| C corrected | Leached AR | Not | 0.16 | −0.40 | −0.05 | −0.03 | −0.05 | −0.5 | N/A^{†} |
| | | Determined | | | | | | | |
| D corrected | Calcined ARG 6S-750 | Not | 0.11 | −0.50 | 0.25 | 0.18 | 0.25 | 0.2 | N/A^{†} |
| | | Determined | | | | | | | |
| E corrected | Leached ARG 6S-750 | Not | 0.12 | 0.0 | 0.05 | −0.025 | −0.05 | 0 | N/A^{†} |
| | | Determined | | | | | | | |

^{†}Vᵢ and Vₜ measured for the substrate samples are within the 95% confidence interval for the mean value so the SARC_{Na} values are considered statistically indistinguishable from the blank mean. Accordingly, a SARC_{Na} determination is not considered applicable for these samples.

### Example CH-4

### A-Glass-SARC_{Na}

A-06F-glass fibers having a mean diameter of 500-600 nm produced by Lauscha Fiber International, is obtained. This glass is used for Samples A, B and C in this example.

A-26F glass sample, as glass fibers having a mean diameter 2.6 micron produced by Lauscha Fiber International, is obtained. This glass is used for Sample D in this example.

Sample A is a sample of as-received A-06F-glass fibers.

Samples B and C are prepared by acid-leach treating the as-received, non-calcined, A-06F-glass. For Samples B and C, about 58.5 g of the A-06F-glass and 4 L 5.5 wt. % nitric acid are each placed in a 4-L wide-neck plastic container. The plastic container is placed in an air draft oven at 90° C. for 2 hr and shaken briefly by hand every 30 minutes. After the acid-leach treatment is completed, the sample is filtered on a Buchner funnel with Whatman 541 paper and washed with about 7.6 L deionized water. Thereafter, the acid-leached sample is dried at 110° C. for 22 hrs.

A-26F glass fibers having mean diameter of about 2.6 microns (2600 nm), produced by Lauscha Fiber International, is obtained. This glass is used, as received, for Sample D.

Samples A-D are analyzed by the Analytical Method for Determining SARC_{Na} described above. The results are presented in the table below.

| | | | Actual Volume of Titrant (ml) Used in Titration | | | | | |
|---|---|---|---|---|---|---|---|---|
| | | | to Obtain pH 9.0, from pH 4.0, at tₒ (Vᵢ) and | | | | | |
| | | Dilute NaOH | to Maintain pH 9.0 at t₅, t₁₀ and t₁₅ (V_{5 to 15}) | | | | | |
| | | Titrant | Vᵢ at | V₅ at | V₁₀ at | V₁₅ at | | |
| Sample ID | Sample Desc. | Conc. (N) | 0 min. | 5 min. | 10 min. | 15 min. | Vₜₒₜₐₗ | Vₜₒₜₐₗ − Vᵢ |
| Blank Mean | Control Mean | 0.01 | 2.1 | 0.15 | 0.125 | 0.15 | 2.5 | N/A |
| A | A-06 as-recv'd | 0.01 | 16.7 | 1.5 | 1.2 | 0.5 | 19.9 | 3.2 |
| B | Leached A-06 | 0.01 | 15.4 | 1.4 | 0.9 | 1.0 | 18.7 | 3.3 |
| C | Leached A-06 | 0.01 | 15.7 | 2.3 | 1.2 | 1.3 | 20.5 | 4.8 |
| D | A-26F as is | 0.01 | 5.4 | 0.7 | 0.5 | 0.3 | 6.9 | 1.5 |

| | | | | Corrected Volume of Titrant (ml) Used in | | | | | |
|---|---|---|---|---|---|---|---|---|---|
| | | | | SARC_{Na} Determination | | | | | |
| | | | S.A._{Kr-BET} | Vᵢ at | V₅ at | V₁₀ at | V₁₅ at | | SARC_{Na} |
| Sample ID | Sample Desc. | IEP | (m²/g) | 0 min. | 5 min. | 10 min. | 15 min. | Vₜₒₜₐₗ | (Vₜₒₜₐₗ − Vᵢ)/Vᵢ |
| Blank Mean | Control Mean | N/A | N/A | 2.1 | 0.15 | 0.125 | 0.15 | 2.5 | N/A |
| A corrected | A-06 as-recv'd | 10.1 | 3.1 | 14.6 | 1.35 | 1.075 | 0.35 | 17.4 | 0.19 |
| B corrected | Leached A-06 | 10.6 | 3.1 | 13.3 | 1.25 | 0.775 | 0.85 | 16.2 | 0.18 |
| C corrected | Leached A-06 | Not | 3.1 | 13.6 | 2.15 | 1.075 | 1.15 | 18.0 | 0.32 |
| | | Determined | | | | | | | |
| D corrected | A-26F as is | Not | <5 | 3.3 | 0.55 | 0.375 | 0.15 | 4.4 | 0.25 |
| | | Determined | | | | | | | |

The catalyst compositions described above are described in further detail in connection with the following example, which illustrates how various catalyst compositions of the type described above can be used in hydrogenation processes. All changes and embodiments that come within the spirit of the invention are intended to be protected. Accordingly, the following examples are not intended to limit the invention described and claimed herein.

### Hydrogenation (HYD) Process Example

In the following non-limiting example, a selected catalyst composition is tested for hydrogenation activity in a laboratory scale plant. The general procedure is described below.

First, catalyst is loaded in a ⅞″ I.D. reactor with a 3/16″ O.D. thermowell. The catalyst is reduced with a H₂ flow rate of 750 cc/min. at 300° C. for about 16 hours.

Second, a hydrocarbon feed composed of 80 wt. % cyclohexane and 20 wt. % benzene is flowed over the catalyst at 72 cc/hr under a pressure of 400 psig. The H₂ to feed molar ratio is about 1.4 to 1. The temperature is varied between 90° C. and 210° C. to achieve the desired conversion of benzene to cyclohexane.

### Example P-1

### HYD W/Platinum on A-Glass

In this example, about 4 g (30 cc) of 0.147 wt. % Pt on A-glass, prepared according to the method described above for Example 25, loaded in the reactor. The catalyst is tested according to the HYD process example procedure described above at a temperature of 207° C.

The results are presented in the table below.

| SAMPLE | | CATALYST | CONVERSION |
|---|---|---|---|
| DESCRIP. | CATALYST | LOADING | (%) |
| Ex. P-1 | 0.147 wt. % Pt | 4.02 g | 11 |
| | on A-Glass | 30 cc | |

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

1. A process comprising hydrogenation of a process stream using a catalyst composition to hydrogenate at least a portion of the process stream, the process stream comprising at least one compound having at least one hydrogenatable site, wherein the catalyst composition comprises:
a substantially nonporous substrate having an external surface, a surface region and a subsurface region,
at least one catalytic constituent, and
at least one catalytically-active region, comprising the at least one catalytic constituent, wherein
(a) the substantially nonporous substrate has
i) a total surface area, as measured by a method selected from the group consisting of S.A._{N2-BET}, S.A._{Kr-BET} and combinations thereof, between about 0.01 m²/g and 10 m²/g; and
ii) a predetermined isoelectric point (IEP) obtained in a pH range greater than 0, but less than or equal to 14;
(b) the at least one catalytically-active region may be contiguous or discontiguous and has
i) a mean thickness less than or equal to about 30 nm; and
ii) a catalytically effective amount of the at least one catalytic constituent; and
(c) the location of the at least one catalytically-active region is substantially
i) on the external surface,
ii) in the surface region,
iii) on the external surface in part and in the surface region in part, or
iv) combinations of (c) (i), (ii) and (iii).

2. The process of claim 1 wherein the at least one catalytic constituent is selected from the group consisting of Bronsted or Lewis acids, Bronsted or Lewis bases, noble metal cations and noble metal complex cations and anions, transition metal cations and transition metal complex cations and anions, transition metal oxy anions, transition metal chalconide anions, main group oxyanions, halides, rare earth ions, rare earth complex cations and anions, noble metals, transition metals, transition metal oxides, transition metal sulfides, transition metal oxysulfides, transition metal carbides, transition metal nitrides, transition metal borides, transition metal phosphides, rare earth hydroxides, rare earth oxides, and combinations thereof.

3. The process of claim 1 wherein, before the composition is under a steady-state hydrogenation reaction condition, the at least one catalytic constituent is a first catalytic constituent having
(a) a first pre-reaction oxidation state and
(b) a first pre-reaction interaction with the substrate selected from the group consisting of ionic charge interaction, electrostatic charge interaction and combinations thereof.

4. The process of claim 3 wherein the first catalytic constituent is selected from the group consisting of acids, bases, chalconides, and combinations thereof.

5. The process of claim 3 wherein, before the composition is under a steady-state hydrogenation reaction condition, at least a portion of the first catalytic constituent is modified or displaced to produce a second catalytic constituent having
(a) a second pre-reaction oxidation state and
(b) a corresponding second pre-reaction interaction with the substrate;
wherein the second pre-reaction oxidation state of the second catalytic constituent is either less than, greater than or equal to the first pre-reaction oxidation state of the first catalytic constituent.

6. The process of claim 5 wherein the second catalytic constituent is selected from the group consisting of Pd, Pt, Rh, Ir, Ru, Os, Cu, Ag, Au, Ru, Re, Ni, Co, Fe, Mn, Cr and combinations thereof.

7. The process of claim 1 wherein the substrate is a glass composition having a SARC_{Na} less than or equal to about 0.5.

8. The process of claim 1 wherein the at least one catalytically-active region is substantially concentrated within a mean thickness less than or equal to about 20 nm.

9. The process of claim 1 wherein the substantially nonporous substrate is selected from the group consisting of AR-glasses, rare earth sodium silicate glasses, silico boroaluminate glasses, E-glasses, boron-free E-glasses, S-glasses, R-glasses, rare earth-silicate glasses, Ba—Ti-silicate glasses, nitrided glasses, A-glasses, C-glasses and CC-glasses and combinations thereof.

10. The process of claim 1 wherein the IEP obtained for the substantially nonporous substrate prior to or after a first leaching treatment is greater than or equal to about 6.0, but less than 14.
